(19) United States
(12) Patent Application Publication
(10) Pub. No.: US 2023/0225308 A1
(43) Pub. Date: Jul. 20, 2023

(54) STABILIZING COMPOSITION AND METHOD FOR PRESERVING A BODILY FLUID

(71) Applicant: DNA Genotek Inc., Kanata, Ontario (CA)

(72) Inventor: Amit Arora, Ottawa, Ontario (CA)

(21) Appl. No.: 17/995,138

(22) PCT Filed: Mar. 30, 2021
(86) PCT No.: PCT/CA2021/050428

§ 371 Date: Sep. 30, 2022

Related U.S. Application Data
(60) Provisional application No. 63/002,123, filed on Mar. 30, 2020.

(51) Int. Cl. A01N 1/02

(52) U.S. Cl. CPC A01N 1/0226

(57) ABSTRACT

An aqueous stabilizing composition for preserving a bodily fluid at ambient temperature is provided. The aqueous stabilizing composition comprises: a sugar selected from a monosaccharide, a disaccharide, or a combination thereof; a buffering agent; a C₁-C₆ alkanol; boric acid, a salt of boric acid, or a combination thereof; and a chelating agent; wherein the composition has a pH of from 4.5 to 5.2. A method for preserving a bodily fluid using the aqueous stabilizing composition is also provided, the method comprising: a) obtaining a sample of the bodily fluid; b) contacting the bodily fluid with the aqueous stabilizing composition to form a mixture; c) mixing the mixture of (b) to form a homogeneous mixture; and d) storing the homogeneous mixture at ambient temperature.

### FIELD OF THE INVENTION

The present invention pertains to a stabilizing composition and method for preserving a bodily fluid at ambient temperature.

### BACKGROUND

Urine, a complex liquid by-product of metabolism in most animals, is used for a variety of analytical tests. In humans, urine consists primarily of water, with organic solutes including urea, creatinine, uric acid, and trace amounts of enzymes, carbohydrates, hormones, fatty acids, pigments, mucin and inorganic ions. Urine, even from healthy individuals, also contains erythrocytes, leukocytes, urothelial cells, renal cells, prostate cells and bacteria. Urine represents a valuable source of biomarkers for the study of urological pathologies due to shedding of cellular and cell-free material from the urogenital apparatus directly into this sample type. Urine from pregnant women is also a useful source of fetal DNA (NBY Tsui, P Jiang, K C K Chow, X Su, T Y Leung, H Sun, K C A Chan, R W K Chiu and Y M D Lo (2012). High resolution size analysis of fetal DNA in the urine of pregnant women by paired-end massively parallel sequencing. PLoS ONE 7(10): e48319) for non-invasive prenatal diagnostic and prognostic tests.

Urinary cell-free DNA (UcfDNA) originates either from cells shedding into urine from the genitourinary tract, or from cell-free DNA (cfDNA) in circulation passing through glomerular filtration. cfDNA exists as fragmented nucleic acids in various extracellular bodily fluids, including urine, in both healthy individuals and people with diseases (e.g. diabetes, cardiovascular diseases, organ transplantation, stroke, epilepsy, autoimmune diseases, sepsis and trauma), serving as an important tool of liquid biopsy (R Meddeb, E Pisareva, A R Thierry (2019) Guidelines for the preanalytical conditions for analyzing circulating cell-free DNA. Clin Chem 65(5): 623-633. Doi: 10.1373/clinchem.2018.298323; C M Stewart, P D Kothari, F Mouliere, R Mair, S Somnay, R Benayed, A Zehir, B Weigelt, S-J Dawson, M E Arcila, M F Berger, D W Y Tsui (2018) The value of cell-free DNA for molecular pathology. J Pathol 244(5): 616-627. Doi: 10.1002/path.5048). UcfDNA is believed to have the potential of being a useful and ultra-non-invasive tool for cancer screening, diagnosis, prognosis, and monitoring of cancer progression and therapeutic efficacy (T Lu and J Li (2017) Clinical applications of urinary cell-free DNA in cancer: Current insights and promising future. Am J Cancer Res 7(11): 2318-2332; S Van Keer, J Pattyn, W A A Tjalma, X Van Ostade, M leven, P Van Damme, A Vorsters (2017) First-void urine: A potential biomarker source for triage of high-risk human papillomavirus infected women. Eur J Obstetrics & Gynecology and Reproductive Biology 216: 1-11). For example, it has recently been reported that first-void urine contains significantly more high risk-human papillomavirus (4.8-160 times) and human DNA than the subsequent fraction (A Vorsters, P Van Damme, G Clifford (2014) Urine testing for HPV: rationale for using first void. BMJ 349: g6252).

Despite growing interest in cell-free DNA (cfDNA) analysis in various clinical fields, especially oncology and prenatal diagnosis, few studies on sample handling have been reported and no analytical consensus is available. Nucleated cells naturally found in urine can release genomic DNA into urine leading to an increased DNA background during sample processing and storage. In addition, enzymatic degradation has the potential to obscure true cfDNA levels, given their relatively small molecular weight. Hence, urine specimens need either special treatment, e.g. processing within a short time of collections (2-4 hours), refrigeration subsequent to collection, or preservation using stabilizing compounds. Given the collection nature of the sample, a preservative is preferably used to preserve the original proportion and integrity of cfDNA in urine post sample collection.

UcfDNA holds great potential as a non-invasive form of liquid biopsy. DNA can be present in both the cellular and cell-free fractions of urine, and the procedures used for collection and processing of DNA will greatly impact the outcome of biomarker analysis (L K Larsen, G E Lind, P Guldberg, C Dahl (2019) DNA-methylation-based detection of urological cancer in urine: Overview of biomarkers and considerations on biomarker design, source of DNA, and detection technologies. Int J Mol Sci 20, 2657). Since cells and DNA in urine are susceptible to degradation upon storage (T H T Cheng, P Jiang, J C W Tam, X Sun, W-S Lee, S C Y Yu, J T C Teoh, P K F Chiu, C-F Ng, K-M Chow, C-C Szeto, K C A Chan, R W K Chiu, Y M D Lo (2017) Whole-genome bisulfite sequencing reveals the origin and time-dependent fragmentation of urinary cfDNA. Clin Biochem 50(9): 496-501. Doi: 10.1016/j.clinbiochem.2017.02.017), proper storage is important when urine samples are not processed immediately. Human urine is a suitable environment for the functioning of nucleic acid-hydrolyzing enzymes (nucleases). Specifically, DNase I is the major DNA-hydrolyzing enzyme in urine and its activity in urine is more than 100-fold higher than its activity in serum (O E Bryzgunova, P P Laktionov (2015) Extracellular nucleic acids in urine: sources, structure, diagnostic potential. Acta Naturae Vol 7(3): 48-54. Doi: 10.32607/20758251-2015-7-3-48-54). The half-life of ucfDNA at body temperature is around 2.6-5.1 hours (THT Cheng et al. (2017) supra). Presently, none of the registered cancer In vitro Diagnostics (IVDs) are based purely on ucfDNA (W J Locke, D Guanzon, C Ma, Y J Liew, K R Duesing, K Y C Fung, J P Ross (2019) DNA methylation cancer biomarkers: translation to the clinic. Front Genet 10: 1150. Doi: 10.3389/fgene.2019.01150). One major reason is because the workflow in preserving ucfDNA has yet to be standardized. Hence, for effective and efficient use of any biochemical and molecular genetic test, the sample collection process, sample transport, sample processing and sample storage/stability should be optimized and standardized.

In healthy individuals, cfDNA originates from apoptosis of nucleated cells (M Stroun, J Lyautey, C Lederrey, A Olson-Sand, P Anker (2001) About the possible origin and mechanism of circulating DNA apoptosis and active DNA release. Clin Chim Acta 313 (1-2): 139-142). In malignancy, the tumor-derived fraction of total cfDNA, termed circulating tumor DNA (ctDNA), can originate from tumor cells by a combination of apoptosis, necrosis and active secretion (M Stroun et al. (2001) supra; S Jahr, H Hentze, S Englisch, D Hardt, F O Fackelmayer, R D Hesch, R Knippers (2001) DNA fragments in the blood plasma of cancer patients: quantitations and evidence for their origin from apoptotic and necrotic cells. Cancer Res 61 (4): 1659-1665; O E Bryzgunova et al. (2015) supra). ctDNA contains tumor-specific mutations, variations in copy number and alterations in DNA methylation status (G Santoni, M B Morelli, C Amantini, N Battelli (2018) Urinary markers in bladder cancer: An update. Front Oncol 8:362. Doi: 10.3389/fonc.2018.00362). ctDNA levels often increase with tumor volume, can be used to predict response to targeted immunotherapies, monitor tumor heterogeneity, and reveal expanding drug resistant tumor clones (R J Diefenbach, J H Lee, R F Kefford, H Rizos (2018) Evaluation of commercial kits for purification of circulating free DNA. Cancer Genetics 228-229: 21-27. Doi: 10.1016/j.cancergen.2018.08.005).

Cancer diagnostics has begun to move away from a sole dependence on direct tumor tissue biopsy for cancer detection, diagnosis, and treatment monitoring. Next-generation sequencing and genomics bioinformatics analysis have brought forth a new paradigm shift from microscopic levels of histologic diagnostics to molecular genomics levels of cancer diagnostics. Novel non-invasive cancer diagnostics platforms, such as liquid biopsy from bodily fluids (i.e., blood, plasma, urine, etc.), are used to interrogate ctDNA or circulating tumor cells, proteomics, metabolomics, and exosomes, which is used to assay ctDNAs (X Wu, L Zhu and PC Ma. Next-generation novel non-invasive cancer molecular diagnostics platforms beyond tissues. Am Soc Clin Oncol Educ Book. 2018 May 23; (38):964-977. Doi: 10.1200/EDBK_199767), among other analytes.

Molecular biomarkers are extensively investigated and may contribute to early detection, monitoring and prediction of therapy response in cancer patients (L Cerchietti and A Melnick (2017) DNA methylation-based biomarkers. J Clin Oncol 35(7):793-795). These biomarkers represent genetic and epigenetic events associated with cancer development and progression. DNA hypermethylation is one example of an epigenetic process. The detection of hypermethylated DNA in bodily fluids, such as urine and blood, are of interest as an oncological biomarker. An important development in cancer care is “liquid biopsy”, which involves the analysis of genetic material of tumor cells shed from primary or metastatic tumors into bodily fluids. A liquid biopsy typically involves extraction and analysis of cfDNA, RNA (miRNA, lncRNAs and mRNAs), proteins, peptides, exosomes or cells derived from biofluids such as blood, urine, saliva and cerebrospinal fluid (A D Meo, J Bartlett, Y Cheng, M D Pasic, G M Yousef (2017) Liquid biopsy: A step forward towards precision medicine in urologic malignancies. Mol Cancer 16: 80. Doi: 10.1186/s12943-017-0644-5). Among the various liquid biopsy samples, urine and saliva are easily obtained without needing an expert for sample collection and enable real-time monitoring of disease through continuous sampling.

Cell-free circulating DNA in blood plasma was first observed in 1948 by Mandel and Metais (P Mandel, P Metais (1948) Les acides nucleiques du plasma sanguine chez l'homme. C R Acad Sci Paris: 241-243). Increased free DNA levels have been shown in the serum and plasma of cancer patients (S A Leon, B Shapiro, D M Sklaroff, M J Yaros (1977) Free DNA in the serum of cancer patients and the effect of therapy. Cancer Res 37: 646-650; S Jahr, et al. (2001) supra). Data from Jahr et al. (2001, supra) is consistent with the possibility that apoptotic and necrotic cells are a major source of plasma DNA in cancer patients. Characteristics of tumour DNA have been found in genetic material extracted from the plasma of cancer patients. These features include decreased strand stability and the presence of specific oncogene, tumour suppressor gene and microsatellite alterations (P Anker, H Mulcahy, X Q Chen, M Stroun (1999) Detection of circulating tumour DNA in the blood (plasma/serum) of cancer patients. Cancer and Metastasis Reviews 18: 65-73. Doi. https://doi.org/10.1023/A:1006260319913). The results obtained in many different cancers indicate that plasma DNA, similar to urine DNA, may be a suitable target for the development of diagnostic, prognostic and follow-up tests for cancer.

The investigation of new biomarkers for renal disease is currently a pressing issue, with renal disease affecting up to 1 in 10 of the US population (J Coresh, E Selvin, L A Stevens, J Manzi, J W Kusek, P Eggers, F Van Lente, A S Levey (2007) Prevalence of chronic kidney disease in the United States. JAMA 298(17): 2038-2047). Urinary extracellular vesicles (UEVs), used in intercellular communication, represent an ideal platform for biomarker discovery (K C Miranda, D T Bond, M McKee, J Skog, T G Paunescu, N Da Silva, D Brown, L M Russo (2010) Nucleic acids within urinary exosomes/microvesicles are potential biomarkers for renal disease. Kidney Int 78(2): 191-199. Doi:10.1038/ki.2010.106). UEVs are small (20-1,000 nm) spherical structures loaded with RNA and protein which are constantly released by healthy and abnormal cells along the entire urogenital tract (A Gamez-Valero, S I Lozano-Ramos, I Bancu, R Lauzurica-Valdemoros, F E Borras (2015) Urinary extracellular vesicles as source of biomarkers in kidney diseases. Front Immunol 6. Doi: http://dx.doi.org/10.3389/fimmu.2015.00006). The term UEVs refers to both plasma membrane-derived (e.g. microvesicles, exosome-like vesicles, ectosomes and retrovirus-like particles) and endosomal-derived vesicles or exosomes. UEVs appear to mirror the physiological condition of the cells of their origin (Gamez-Valero et al. (2015), supra; D Tataruch-Weinert, L Musante, O Kretz, H Holthofer (2016) Urinary extracellular vesicles for RNA extraction: optimization of a protocol devoid of prokaryote contamination. J Extracellular Vesicles 5: 30281—http://dx.doi.org/10.3402/jev.v5.30281). Additionally, secreted vesicles mediate specific aspects of inter-cellular communication by their miRNA, mRNA and tRNA known as “exosomal shuttle RNA” (H Valadi, K Ekstrom, A Bossios, M Sjostrand, J J Lee, L O Lotvall (2007) Exosome-mediated transfer of mRNAs and microRNAs is a novel mechanism of genetic exchange between cells. Nature Cell Biology 9: 654-659). Depending on the urine collection method, UEVs enrichment and RNA extraction methods, marked variability has been observed in reported RNA profiles (D Tataruch-Weinert et al. (2016), supra).

Recent studies suggest that EVs may be the key to timely diagnosis and monitoring of genito-urological malignancies. Urine exosomes, a subclass of EVs, are small vesicles that contain proteins, mRNAs and microRNAs (miRNAs) and are released by cells in all segments of the nephron and the urogenital tract. Exosomes produced by prostate cells travel with prostate secretions via prostate ejaculatory ducts that empty directly into the urethra and pass into the urine where they can be readily detected (O E Bryzgunova, M M Zaripov, T E Skvortsova, E A Lekchnov, A E Grigor'eva, I A Zaporozhchenko, E S Morozkin, E I Ryabchikova, Y B Yurchenko, V E Voitsitskiy, P P Laktionov (2016) Comparative study of extracellular vesicles from the urine of healthy individuals and prostate cancer patients. PLoS ONE 11(6): e0157566. Doi:10.1371/journal.pone.0157566). Nilsson et al. (J Nilsson, J Skog, A Nordstrand, V Baranov, L Mincheva-Nilsson, X O Breakefield, A Widmark (2009) Prostate cancer-derived urine exosomes: a novel approach to biomarkers for prostate cancer. Br J Cancer 100: 1603-1607. Doi: 10.1038/sj.bjc.6605058) were able to detect two known prostate cancer mRNA biomarkers, PCA3 and TMPRSS2-ERG, in exosomes isolated from the urine of prostate cancer patients, showing the potential of EVs for use in prostate cancer diagnostics. This and other studies support the use of RNA in exosomes isolated from urine as diagnostic markers for prostate cancer, and offers an alternative, sensitive and unique new type of screening for cancer biomarkers.

For urological cancers, urine is in many situations the preferred liquid biopsy source because it contains exfoliated tumor cells and cell-free tumor DNA and can be obtained easily, noninvasively, and repeatedly (L K Larsen, G E Lind, P Guldberg, C Dahl (2019) DNA-methylation-based detection of urological cancer in urine: Overview of biomarkers and considerations on biomarker design, source of DNA, and detection technologies. Int J Mol Sci 20, 2657). Compared to blood, urine is thought to be a more sensitive alternative for early detection or monitoring recurrence of cancers in the genitourinary tract (S Y Lin, J A Linehan, T G Wilson, D S B Hoon (2017) Emerging utility of urinary cell-free nucleic acid biomarkers for prostate, bladder, and renal cancers. Eur Urol Focus 3(2-3): 265-272. Doi: 10.1016/j.euf.2017.03.009). In addition, there is no need for qualified personnel to obtain the sample which allows for collection at home. However, the utilization of urinary hypermethylated DNA in clinical practice is constrained by the challenges of preserving urinary nucleic acids. Hence, urine needs to be stored and transported in such a way that nucleic acid preservation is ensured to allow for downstream analysis (J Bosschieter, S Bach, I V Bijnsdorp, L I Segerink, W F Rurup, A P van Splunter, I Bahce, P W Novianti, G Kazemier, R J A van Moorselaar, R D M Steenbergen, J A Nieuwenhuijzen (2018) A protocol for urine collection and storage prior to DNA methylation analysis. PLoS ONE 13(8): e0200906).

There is a need for stabilizing compositions for preserving bodily fluids, such as urine, at ambient temperature.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

While multiple commercial products for nucleic acid stabilization in biological samples such as bodily fluids exist, these are largely intended for stabilizing either DNA or RNA, but not both simultaneously. A composition to efficiently stabilize both cellular and cell-free nucleic acids in bodily fluids, such as urine, has not yet been reported. It would be beneficial to provide a collection device and composition located therein that prevents the lysis of intact bacterial and human cells, thereby blocking the release of unwanted nucleic acids into the biological sample which would otherwise contaminate the in vivo urinary signal. The composition would additionally prevent the release of membrane vesicles. This is critical as cell-free RNA is encapsulated in membrane vesicles, including microvesicles and extracellular vesicles (including, but not limited to exosomes). Preferably, the composition would maintain stability and integrity of both cell-free and cellular nucleic acids (DNA and RNA) in a bodily fluid, such as urine, for a minimum of 7 days at room temperature, preventing both chemical- and enzymatic-based degradation. The present application discloses such a composition.

In one aspect, there is provided an aqueous stabilizing composition for preserving a bodily fluid at ambient temperature, the composition comprising: a sugar selected from a monosaccharide, a disaccharide, or a combination thereof; a buffering agent; a C₁-C₆ alkanol; boric acid, a salt of boric acid, or a combination thereof; and a chelating agent; wherein the composition has a pH of from 4.5 to 5.2.

In another aspect, there is provided a method for preserving a bodily fluid, the method comprising: a) obtaining a sample of the bodily fluid; b) contacting the bodily fluid with an aqueous stabilizing composition to form a mixture, the composition comprising: a sugar selected from a monosaccharide, a disaccharide, or a combination thereof; a buffering agent; a C₁-C₆ alkanol; boric acid, a salt of boric acid, or a combination thereof; and a chelating agent; wherein the composition has a pH of from 4.5 to 5.2; c) mixing the mixture of (b) to form a homogeneous mixture; and d) storing the homogeneous mixture at ambient temperature.

In yet another aspect, there is provided an aqueous composition comprising: a sugar selected from a monosaccharide, a disaccharide, or a combination thereof; a buffering agent; a C₁-C₆ alkanol; boric acid, a salt of boric acid, or a combination thereof; a chelating agent; and a bodily fluid.

### BRIEF DESCRIPTION OF THE FIGURES

For a better understanding of the present invention including the progression of development to get to the end product, reference is made to the following description which is to be used in conjunction with the accompanying drawings, where:

FIG. 1 is a chart illustrating urinary cell-free DNA (UcfDNA) from female and male donors, which shows that the amount of UcfDNA in urine samples is both sample and sex-dependent.

FIG. 2A is a chart illustrating increasing turbidity of a non-stabilized first morning, first void (FMFV) urine sample due to bacterial growth (as evidenced further by FIG. 2B).

FIG. 2B is a chart illustrating ΔCₜ [C_{t(T7)}-C_{t(T0)}] determined from bacterial 16S and β-globin qPCR assay for the quantification of bacterial and human cell-free DNA (cfDNA) content in unstabilized urine samples after 7 days at RT (room temperature).

FIGS. 2C and 2D illustrate results of Agilent 4200 Tapestation analysis, showing a massive decline in human cell-free DNA content after 7 days at room temperature.

FIGS. 3A, 3B and 3C are charts illustrating (i) stability and (ii) neutrality as ΔCₜ [C_{t(T7)}-C_{t(T0)}] and ΔCₜ [C_{t(T0 Chem)}-C_{t(T0 NA)}], respectively, and determined from β-globin qPCR assay for the quantification of human cfDNA content in urine samples at day 0, as well as after storage at RT for 7 days under various conditions, including in admixture with the aqueous stabilizing compositions of the present application. C_{t (T0)} and C_{t (T7)} denotes qPCR cycle threshold at day 0 and day 7, respectively. C_{t(T0 Chem)} and C_{t(T0 NA)} denotes qPCR cycle threshold for urine specimen with chemistry and unpreserved specimen (NA), respectively at day 0.

FIGS. 4A and 4B are charts illustrating ΔCₜ [C_{t(T)}-C_{t(T0 NA)}] determined from β-globin qPCR assay for the quantification of human cfDNA content in urine samples after storage at room temperature for 7 days under various conditions, including in admixture with the aqueous stabilizing composition of the present application. C_{t(T)} denotes qPCR cycle threshold at day 7. C_{t(T0 NA)} denotes qPCR cycle threshold for unpreserved specimen (NA) at day 0

FIG. 5A illustrates (i) stability and (ii) neutrality as ΔCₜ [C_{t(T7)}-C_{t(T0)}] and ΔCₜ [C_{t(T0 Chem)}-C_{t(T0 NA)}], respectively, and determined from β-globin qPCR assay for the quantification of human cfDNA content in urine samples at day 0, as well as after storage at RT for 7 days under various conditions, including in admixture with the aqueous stabilizing composition of the present application. C_{t (T0)} and C_{t (T7)} denotes qPCR cycle threshold at day 0 and day 7, respectively. C_{t(T0 Chem)} and C_{t(T0 NA)} denotes qPCR cycle threshold for urine specimen with chemistry and unpreserved specimen (NA), respectively at day 0. FIG. 5B illustrates a representative Tapestation profile analysis of these unpreserved and Chemistry F (Chem F) containing urine samples, showing that cfDNA is degraded in unpreserved samples and is stabilized in the aqueous stabilizing composition of the present application. FIGS. 5C, 5D and 5E are charts illustrating (i) stability and (ii) neutrality as ΔCₜ [C_{t(T)}-C_{t(T0)}] and ΔCₜ [C_{t(T0 Chem)}-C_{t(T0 NA)}], respectively, and determined from β-globin qPCR assay for the quantification of human cfDNA content in urine samples at day 0, as well as after storage at RT for 7 or 14 days under various conditions, including in admixture with the aqueous stabilizing composition of the present application. C_{t (T0)} and C_{t (T)} denotes qPCR cycle threshold at day 0 and day 7 or day 14, respectively. C_{t(T0 Chem)} and C_{t(T0 NA)} denotes qPCR cycle threshold for urine specimen with chemistry and unpreserved specimen (NA), respectively at day 0.

FIG. 6(i) A & B are charts illustrating ΔCₜ [C_{t(T)}-C_{t(T0NA)}] determined from β-globin qPCR assay for the quantification of human cfDNA content in urine samples spiked (S) with prostate cancer cells at day 0, as well as after storage at RT for 7 days under various conditions, including in admixture with the aqueous stabilizing composition of the present application. C_{t(T)} denotes qPCR cycle threshold at day 0 or day 7. C_{t(T0 NA)} denotes qPCR cycle threshold for unpreserved spiked specimen (NA) at day 0. FIG. 6(i)C illustrates representative Tapestation profile analysis of the unpreserved and Chemistry F (Chem F) containing urine samples. FIG. 6(ii)A is a chart illustrating ΔCₜ [C_{t(T)}-C_{t(T0NA)}] determined from β-globin qPCR assay for the quantification of human cfDNA content in urine samples spiked (S) with prostate cancer cells at day 0, as well as after storage at RT for 7 days under various conditions, including in admixture with the aqueous stabilizing composition of the present application. C_{t(T)} denotes qPCR cycle threshold at day 0 or day 7. C_{t(T0 NA)} denotes qPCR cycle threshold for unpreserved spiked specimen (NA) at day 0. FIG. 6(ii)B is a chart illustrating the number of copies of β-globin gene per unit volume for some of these samples determined using ddPCR assay. Collectively FIGS. 6(i) and (ii) suggest that the aqueous stabilizing composition of the present application preserves the integrity of prostate cancer cells in a concentration-dependent manner at room temperature for at least 7 days.

FIG. 7 is a chart illustrating ΔCₜ [C_{t(T)}-C_{t(T0 NA)}] determined from β-globin qPCR assay for the quantification of human cfDNA content in urine samples spiked (S) with nucleated white blood cells at day 0, as well as after storage at RT for 7 days under various conditions, including in admixture with the aqueous stabilizing composition of the present application, as well as a commercially available composition from Streck. C_{t(T)} denotes qPCR cycle threshold at day 0 or day 7. C_{t(T0 NA)} denotes qPCR cycle threshold value for unpreserved spiked specimen (NA) at day 0.

FIG. 8A shows HpaII and MspI restriction endonuclease digestion pattern confirming in vitro plasmid DNA methylation using CpG Methyl Transferase. FIGS. 8B and 8C shows Tapestation results for PCR amplification of methylated plasmid, suggesting preservation of DNA methylation in the present composition for 7 days at RT.

FIGS. 9A and 9B are charts illustrating ΔCₜ [C_{t(T7)}-C_{t(T0)}] determined from Ampicillin resistance gene (Amp^{R}) and bacterial 16S qPCR assay for the respective quantification of HPV plasmid DNA and bacterial DNA content in both the unpreserved and Chemistry F (Chem F) containing urine samples spiked with purified HPV16 plasmid DNA after storage at room temperature for 7 days. C_{t(T7)} denotes qPCR cycle threshold at day 7. C_{t(T0)} denotes qPCR cycle threshold at day 0.

FIG. 10A illustrates (i) stability and (ii) neutrality as ΔCₜ [C_{t(T7)}-C_{t(T0)}] and ΔCₜ [C_{t(T0 Chem)}-C_{t(T0 NA)}], respectively, and determined from β-actin RT-qPCR assay for the quantification of human EV RNA content in urine samples at day 0, as well as after storage at RT for 7 days under various conditions, including in admixture with the aqueous stabilizing composition of the present application. C_{t (T0)} and C_{t (T7)} denotes qPCR cycle threshold at day 0 and day 7, respectively. C_{t(T0 Chem)} and C_{t(T0 NA)} denotes qPCR cycle threshold for urine specimen with chemistry and unpreserved specimen (NA), respectively at day 0. FIG. 10B illustrates representative electropherogram traces of extracellular vesicles (EV) RNA from both unpreserved and Chemistry F (Chem F) containing urine specimens at day 0 and day 7. FIGS. 10C and 10D illustrate (i) stability and (ii) neutrality as ΔCₜ [C_{t(T7)}-C_{t(T0)}] and ΔCₜ [C_{t(T0 Chem)}-C_{t(T0 NA)}], respectively, and determined from β-actin RT-qPCR assay for the quantification of human EV RNA content in urine samples at day 0, as well as after storage at RT for 7 days under various conditions, including in admixture with the aqueous stabilizing composition of the present application. C_{t (T0)} and C_{t (T7)} denotes qPCR cycle threshold at day 0 and day 7, respectively. C_{t(T0 Chem)} and C_{t(T0 NA)} denotes qPCR cycle threshold for urine specimen with chemistry and unpreserved specimen (NA), respectively at day 0.

FIG. 11 is a chart illustrating (i) stability and (ii) neutrality as ΔCₜ [C_{t(T7)}-C_{t(T0)}] and ΔCₜ [C_{t(T0 Chem)}-C_{t(T0 NA)}], respectively, and determined from p-actin RT-qPCR assay for the quantification of human cell free RNA content in urine samples at day 0, as well as after storage at RT for 7 days under various conditions, including in admixture with the aqueous stabilizing composition of the present application. C_{t (T0)} and C_{t (T7)} denotes qPCR cycle threshold at day 0 and day 7, respectively. C_{t(T0 Chem)} and C_{t(T0 NA)} denotes qPCR cycle threshold for urine specimen with chemistry and unpreserved specimen (NA), respectively at day 0.

FIGS. 12A and 12B are charts illustrating (i) stability and (ii) neutrality as ΔCₜ [C_{t(T7)}-C_{t(T0)}] and ΔCₜ [C_{t(T0 Chem)}-C_{t(T0 NA)}], respectively, and determined from p-actin RT-qPCR assay for the quantification of cellular RNA content in urine samples at day 0, as well as after storage at RT for 7 days under various conditions, including in admixture with the aqueous stabilizing composition of the present application. C_{t (T0)} and C_{t (T7)} denotes qPCR cycle threshold at day 0 and day 7, respectively. C_{t(T0 Chem)} and C_{t(T0 NA)} denotes qPCR cycle threshold for urine specimen with chemistry and unpreserved specimen (NA), respectively at day 0.

FIG. 13A illustrates the Tapestation profile of day 0 and day 7 extracted cellular DNA in both unpreserved (NA) and Chemistry F (Chem F) containing urine specimens admixed with the aqueous stabilizing composition of the present application. FIG. 13B shows Tapestation profile of PCR amplified GAPDH product. FIG. 13C illustrates % bacterial DNA content determined from bacterial 16S qPCR assay.

FIG. 14 illustrates the Tapestation profile of day 0 and day 7 extracted cfDNA in both unpreserved (TE) and Chemistry F containing saliva specimens admixed with the aqueous stabilizing composition of the present application. TE stands for 1× Tris-EDTA buffer.

### DETAILED DESCRIPTION OF THE INVENTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) ingredient(s) and/or elements(s) as appropriate.

Terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

The term “bodily fluid” as used herein will be understood to mean a naturally occurring fluid from a human or an animal, and includes, but is not limited to urine, saliva, sputum, serum, plasma, blood, pharyngeal, nasal/nasal pharyngeal and sinus secretions, mucous, gastric juices, pancreatic juices, bone marrow aspirates, cerebral spinal fluid, feces, semen, products of lactation or menstruation, cervical secretions, vaginal fluid, tears, or lymph. In one embodiment, the bodily fluid is selected from urine or saliva. In another embodiment, the bodily fluid is urine.

The term “ambient temperature” as used herein refers to a range of temperatures that could be encountered by the mixture of a bodily fluid (e.g. urine sample) and the aqueous stabilizing composition described herein from the point of collection, during transport (which can involve relatively extreme temperatures, albeit usually for shorter periods of time (e.g. <5 days)), as well as during prolonged storage prior to analysis. In one embodiment, the ambient temperature is ranging from about −20° C. to about 50° C. In another embodiment, the ambient temperature is room temperature (RT) and ranges from about 15° C. to about 25° C.

The term “monosaccharide” as used herein will be understood to mean a sugar that is not decomposable into simpler sugars by hydrolysis, is classed as either an aldose or ketose, and contains one or more hydroxyl groups per molecule. In one embodiment, the monosaccharide is selected from fructose, glucose, mannose, or galactose. In another embodiment, the monosaccharide is fructose, glucose, or a combination thereof.

The term “disaccharide” as used herein will be understood to mean a compound in which two monosaccharide units are joined by a glycosidic linkage. In one embodiment, the disaccharide is selected from sucrose, trehalose, and lactose. In another embodiment, the disaccharide is sucrose.

It has been found that compositions according to the present application comprising disaccharides can be more difficult to prepare, as such solutions may have very high viscosities which can lead to improper mixing of the components and/or addition to the specimen (i.e. bodily fluid) due to difficulties in mixing. Overall, due to workability of the samples, monosaccharides are preferred over disaccharides for the compositions and methods of the present application.

The term “chelator” or “chelating agent” as used herein will be understood to mean a chemical that will form a soluble, stable complex with certain metal ions (e.g., Ca²⁺ and Mg²⁺), sequestering the ions so that they cannot normally react with other components, such as deoxyribonucleases (DNases) or endonucleases (e.g. type I, II and III restriction endonucleases) and exonucleases (e.g. 3′ to 5′ exonuclease), enzymes which are abundant in various body fluid samples. In the present composition, chelating agent(s) participates in the inhibition of DNases and microbial growth in biological samples. A chelator can be, for example, ethylene glycol tetraacetic acid (EGTA), (2-hydroxyethyl)ethylenediaminetriacetic acid (HEDTA), diethylene triamine pentaacetic acid (DTPA), nitrilotriacetic acid (NTA), ethylenediaminetriacetic acid (EDTA), 1,2-cyclohexanediaminetetraacetic acid (CDTA), N,N-bis(carboxymethyl)glycine, triethylenetetraamine (TETA), tetraazacyclododecanetetraacetic acid (DOTA), desferioximine, citrate anhydrous, sodium citrate, calcium citrate, ammonium citrate, ammonium bicitrate, citric acid, diammonium citrate, ferric ammonium citrate, and lithium citrate. These chelating agents may be used singly or in combination of two or more thereof.

The term “C₁-C₆ alkanol” as used herein will be understood to mean straight-chain or branched, such as methanol, ethanol, propanol, isopropanol, butanol, n-butanol, pentanol, hexanol, or any combination thereof. In one embodiment of the present composition, the preferred alcohol is ethanol.

In one embodiment, there is provided an aqueous stabilizing composition for preserving a bodily fluid at ambient temperature, the composition comprising: a sugar selected from a monosaccharide, a disaccharide, or a combination thereof; a buffering agent; a C₁-C₆ alkanol; boric acid, a salt of boric acid, or a combination thereof; and a chelating agent; wherein the composition has a pH of from 4.5 to 5.2.

In one embodiment, the aqueous composition comprises boric acid; a salt of boric acid, such as, for example, dihydrogen borate, hydrogen borate, diborate, triborate, tetraborate, metaborate, hydroxoborate, borate salts; or a combination thereof. In another embodiment, the aqueous composition comprises boric acid, sodium borate, or a combination thereof. In yet another embodiment, the aqueous composition comprises boric acid. In one embodiment, the boric acid, the salt of boric acid or the combination thereof is present in the aqueous stabilizing composition in an amount of from about 0.5% to about 5% (wt/vol), or from about 1% to about 3% (wt/vol); or from about 2% to about 2.5% (wt/vol), or about 2.2% (wt/vol).

In one embodiment, the sugar is a monosaccharide, such as, for example, fructose, glucose, mannose, galactose, or a combination thereof. In another embodiment, the monosaccharide is fructose, glucose, or a combination thereof. In another embodiment, the sugar is a disaccharide, such as, for example, trehalose, lactose, or sucrose, or a combination thereof. In another embodiment, the disaccharide is sucrose. In one embodiment, the sugar is present in the aqueous stabilizing composition in an amount of from about 5% to about 45% (wt/vol), of from about 5% to about 40% (wt/vol), or from about 10% to about 30% (wt/vol), or from about 18% to about 22% (wt/vol), or about 20% (wt/vol).

In general, the pH of the present aqueous stabilizing composition can be maintained in the desired range using one or more appropriate buffering agents. In accordance with one embodiment, the composition comprises one, two, or more buffering agents (non-limiting examples being acetate buffer and citrate buffer, such as sodium acetate, potassium acetate, ammonium acetate, sodium citrate, and ammonium citrate) with pKₐ values, logarithmic acid dissociation constants, at 25° C. ranging from 3 to 6.5 to maintain the pH within the preferred range of 4.5 to 5.2. In one embodiment, the buffering agent is sodium acetate.

An acid dissociation constant, Kₐ, is a quantitative measure of the strength of an acid in solution. The larger the Kₐ value, the more dissociation of the molecules in solution and thus the stronger the acid. Due to the many orders of magnitude spanned by Kₐ values, a logarithmic measure of the acid dissociation constant, pKₐ, is more commonly used in practice. The larger the value of pKₐ, the smaller the extent of dissociation at any given pH, i.e., the weaker the acid. In living organisms, acid-base homeostasis and enzyme kinetics are dependent on the pKₐ values of many acids and bases present in the cell and in the body. In chemistry, knowledge of pKₐ values is necessary for the preparation of buffer solutions and is also a prerequisite for a quantitative understanding of the interaction between acids or bases and metal ions to form complexes. One skilled in the art will understand that a given compound/buffer can buffer the pH of a solution only when its concentration is sufficient and when the pH of the solution is close (within about one pH unit) to its pKₐ. In one embodiment, the pH of the present composition is in the range of 4.5 to 5.2. In a preferred embodiment, the pH of the composition is about 5.0. The amount of buffering agent(s) in the aqueous stabilizing composition can be of from about 150 mM to about 1.75 M, or from about 150 mM to about 1.5 M, or from about 500 mM to about 1.2 M, or from about 0.7 M to about 0.8 M, or about 0.75 M, for example.

In one embodiment, the C₁-C₆ alkanol in the aqueous stabilizing composition is selected from methanol or ethanol. In another embodiment, the C₁-C₆ alkanol is ethanol. In yet another embodiment, the C₁-C₆ alkanol is present in the aqueous stabilizing composition in an amount of from about 5% to about 50% (vol/vol), or from about 10% to about 30% (vol/vol), or from about 20% to about 25% (vol/vol), or about 23% (vol/vol).

Ethanol causes dehydration of proteins or a reduction in water activity, followed by electrostatic attraction between proteins, aggregation and insolubilization. While wishing to not be bound by theory, the inventor believes that ethanol, at the percentage used, has little to no fixative properties in this composition; rather, it is important for overall stability and enhances the functionality of other chemical compounds which may be included in the present composition. In addition, for shipping/transport of flammable liquids, it is desirable to keep organic solvents, such as ethanol, below 24% by volume in solutions for exemption from Transport of Dangerous Goods (TDG) regulations (United Nations (UN) number 1170); otherwise a solution with >24% ethanol is classified as class 3 (flammable liquids), special packaging is mandated, and transport complexity and costs increase. As such, an aqueous stabilizing composition comprising about 23% (vol/vol) or lower is particularly advantageous.

In another embodiment, the chelating agent in the aqueous stabilizing composition is selected from, for example, ethylenediaminetriacetic acid (EDTA), 1,2-cyclohexanediamine tetraacetic acid (CDTA), diethylenetriamine pentaacetic acid (DTPA), tetraazacyclododecanetetraacetic acid (DOTA), tetraazacyclotetradecanetetraacetic acid (TETA), desferioximine, or chelator analogs thereof. In another embodiment, the chelating agent is CDTA. In another embodiment, the chelating agent is present in the aqueous stabilizing composition in an amount of from about 10 mM to about 120 mM, or from about 10 mM to about 100 mM, or from about 30 mM to about 70 mM, or from about 40 mM to about 60 mM, or about 50 mM.

In one embodiment of the aqueous stabilizing composition, the composition comprises, consists essentially of, or consists of: the sugar (such as fructose, glucose, sucrose, or a combination thereof; preferably fructose, glucose, or a combination thereof) in an amount of from about 5% to about 45% (wt/vol), of from about 5% to about 40% (wt/vol), or from about 10% to about 30% (wt/vol), or from about 18% to about 22% (wt/vol), or about 20% (wt/vol); the buffering agent (such as, for example, sodium acetate) in an amount of from about 150 mM to about 1.75 M, or from about 150 mM to about 1.5 M, or from about 500 mM to about 1.2 M, or from about 0.7 M to about 0.8 M, or about 0.75 M; the C₁-C₆ alkanol (such as methanol, ethanol, or a combination thereof; preferably ethanol) in an amount of from about 5% to about 50% (vol/vol), or from about 10% to about 30% (vol/vol), or from about 20% to about 25% (vol/vol), or about 23% (vol/vol); the boric acid, the salt of boric acid or the combination thereof (preferably boric acid) in an amount of from about 0.5% to about 5% (wt/vol); or from about 1% to about 3% (wt/vol); or from about 2% to about 2.5% (wt/vol), or about 2.2% (wt/vol); and the chelating agent (such as CDTA) in an amount of from about 10 mM to about 120 mM, or from about 10 mM to about 100 mM, or from about 30 mM to about 70 mM, or from about 40 mM to about 60 mM, or about 50 mM.

In one embodiment, the aqueous stabilizing composition stabilizes cells (such as cancer cells or nucleated blood cells), extracellular vesicles, nucleic acids (e.g. cellular DNA and RNA, such as cell-free DNA (cfDNA), cell-free RNA (cfRNA), and extracellular vesicle RNA (EV RNA)), and/or microorganisms (such as bacteria or viruses) contained in the bodily fluid.

In another embodiment, there is provided a method for preserving a bodily fluid, the method comprising: a) obtaining a sample of the bodily fluid; b) contacting the bodily fluid with the aqueous stabilizing composition as defined above to form a mixture; c) mixing the mixture of (b) to form a homogeneous mixture; and d) storing the homogeneous mixture at ambient temperature. In one embodiment, preserving the bodily fluid comprises stabilizing cells (such as cancer cells or nucleated blood cells), extracellular vesicles, nucleic acids (e.g. DNA and RNA, such as cell-free DNA (cfDNA), cell-free RNA (cfRNA), and extracellular vesicle RNA (EV RNA)), and/or microorganisms (such as bacteria or viruses) contained in the bodily fluid. In another embodiment, the cells, nucleic acids, extracellular vesicles, and/or microorganisms contained in the bodily fluid are stabilized for at least 7 days at ambient temperature. In another embodiment, the cells, nucleic acids, extracellular vesicles, and/or microorganisms contained in the bodily fluid are stabilized for at least 14 days at ambient temperature. In another embodiment, the bodily fluid is urine or saliva. In another embodiment, the bodily fluid is urine.

In yet another embodiment, there is provided an aqueous composition comprising: a sugar selected from a monosaccharide, a disaccharide, or a combination thereof; a buffering agent; a C₁-C₆ alkanol; boric acid, a salt of boric acid, or a combination thereof; a chelating agent; and a bodily fluid. In one embodiment, the bodily fluid is urine. In another embodiment, the bodily fluid is urine and the pH of the aqueous composition comprising the bodily fluid is between 5 and 5.5. In another embodiment, the sugar is present in an amount of from about 1.5% to about 15% (wt/vol), or from about 2% to about 10% (wt/vol), or from about 5% to about 7% (wt/vol), or about 6% (wt/vol); the buffering agent is present in an amount of from about 50 mM to about 500 mM, or from about 200 mM to about 400 mM, or from about 220 mM to about 240 mM, or about 230 mM, or about 225 mM; the C₁-C₆ alkanol is present in an amount of from about 2% to about 40% (vol/vol), or from about 3% to about 20% (vol/vol), or from about 5% to about 10% (vol/vol), or about 6.5% (vol/vol), or about 6.9% (vol/vol); the boric acid, the salt of boric acid or the combination thereof is present in an amount of from about 0.1% to about 2% (wt/vol), or from about 0.2% to about 1.5% (wt/vol), or from about 0.5% to about 1.0% (wt/vol), or about 0.7% (wt/vol), or about 0.6% (wt/vol), and the chelating agent is present in an amount of from about 2.5 mM to about 50 mM, or from about 5 mM to about 25 mM, or from about 10 mM to about 20 mM, or about 16 mM, or about 15 mM.

In one embodiment, the bodily fluid is urine and the urine sample is collected using a device for capturing a predetermined volume of a predefined portion of urine (e.g. first void), such as that described in WO2014037152 entitled “LIQUID SAMPLER, KIT OF PARTS, AND METHOD FOR ASSEMBLY”. In one embodiment, the Colli-Pee® First Void Urine Collection Device (Novosanis) can be used. The aqueous stabilizing composition can be present in the device at the time of collection, or the urine can be contacted with the aqueous stabilizing composition immediately post-collection. The reservoir containing the urine sample and aqueous stabilizing composition can be sealed with an appropriate cap, and the combined sample and stabilizing composition can be gently mixed, for example by inverting the tube. Urine samples can also be collected in standard urine specimen containers (e.g. VWR, Cat. No. 10804-050) and then mixed with the stabilizing composition. Alternatively, collected urine can be transported to the laboratory on ice packs where it can be mixed with the present stabilizing composition.

In another embodiment, the bodily fluid is saliva and the saliva sample is collected using a device such as, for example, those described in WO2007/068094 entitled “CONTAINER SYSTEM FOR RELEASABLY STORING A SUBSTANCE”, WO2010/020043 entitled “SAMPLE RECEIVING DEVICE”, and WO2010/130055 entitled “CLOSURE, CONTAINING APPARATUS, AND METHOD OF USING SAME”.

In another embodiment, the bodily fluid is feces, and the fecal sample is collected using a device such as that described in WO2015172250 entitled “DEVICE FOR COLLECTING, TRANSPORTING AND STORING BIOMOLECULES FROM A BIOLOGICAL SAMPLE”.

In still another embodiment, the sample of the bodily fluid can be collected in a standard, commercially-available laboratory or transport tube (e.g. 10 mL round-bottom tube (92×15.3 mm), Cat. No. 60.610; Sarstedt, or larger tube depending on the sample type and size). The tube containing the sample of the bodily fluid and aqueous stabilizing composition can be sealed with an appropriate cap, and the combined sample and stabilizing composition can be gently mixed, for example by inverting the tube.

Bodily fluid should preferably be mixed immediately with the stabilizing composition at the point of collection. Otherwise, samples should be stored and/or transported on ice packs or refrigerated before mixing with the composition.

As the skilled worker will appreciate, the aqueous stabilizing composition (“chemistry”) described herein can be combined with the sample of the bodily fluid in a variety of ratios. For example, where the bodily fluid is urine, it is desirable to avoid overly diluting the sample and thus reducing the analytes collected; thus, the ratio of chemistry:urine can range, for instance, from 0.25:1 to 0.75:1—e.g. 0.25:1, 0.30:1, 0.35:1, 0.40:1, 0.45:1, 0.50:1, 0.55:1, 0.60:1, 0.65:1, 0.70:1, or 0.75:1. In one embodiment, the ratio of chemistry:urine is 0.40:1 to 0.45:1.

For other bodily fluids, such as feces, in order to ensure sufficient mixing, higher ratios of chemistry:sample can be used.

In one embodiment, following the step of contacting the bodily fluid with the aqueous stabilizing composition and mixing to form a homogeneous mixture, the homogenous mixture then comprises: the sugar (such as fructose, glucose, sucrose, or a combination thereof; preferably fructose, glucose, or a combination thereof) in an amount of from about 1.5% to about 15% (wt/vol), or from about 2% to about 10% (wt/vol), or from about 5% to about 7% (wt/vol), or about 6% (wt/vol); the buffering agent (such as, for example, sodium acetate) in an amount of from about 50 mM to about 500 mM, or from about 200 mM to about 400 mM, or from about 220 mM to about 240 mM, or about 230 mM, or about 225 mM; the C₁-C₆ alkanol (such as methanol, ethanol, or a combination thereof; preferably ethanol) in an amount of from about 2% to about 40% (vol/vol), or from about 3% to about 20% (vol/vol), or from about 5% to about 10% (vol/vol), or about 6.5% (vol/vol), or about 6.9% (vol/vol); the boric acid, the salt of boric acid or the combination thereof (preferably boric acid) in an amount of from about 0.1% to about 2.2% (wt/vol); or from about 0.2% to about 1.5% (wt/vol); or from about 0.5% to about 1.0% (wt/vol), or about 0.7% (wt/vol) or about 0.6% (wt/vol); and the chelating agent (preferably CDTA) in an amount of from about 2.5 mM to about 50 mM, or from about 5 mM to about 25 mM, or from about 10 mM to about 20 mM, or about 16 mM, or about 15 mM.

As noted above, in one embodiment, the aqueous stabilizing composition stabilizes cells (such as cancer cells or nucleated blood cells), extracellular vesicles, nucleic acids (e.g. DNA and RNA, such as cell-free DNA (cfDNA), cell-free RNA (cfRNA), and extracellular vesicle RNA (EV RNA)), and/or microorganisms (such as bacteria or viruses) contained in the bodily fluid. In one embodiment, the aqueous stabilizing composition stabilizes such components of the bodily fluid for at least 7 days at ambient temperature. In another embodiment, the aqueous stabilizing composition stabilizes such components of the bodily fluid for at least 14 days at ambient temperature. Such stabilization can be assessed by methods known to those skilled in the art, such as via monitoring the degradation of cell-free nucleic acids (described further in the Materials and Methods section, and in the Examples which follow).

ΔCₜ corresponds to the relative change in the amount or expression of a given gene. ΔCₜ corresponds to C_{t(T)}-C_{t(T0)}, where C_{t(T)} stands for cycle threshold at day 7 or day 14 while C_{t(T0)} denotes cycle threshold at day 0. Cycle threshold (Cₜ) value of a reaction is defined as the cycle number when the fluorescence of a PCR product can be detected above the background signal. In the present studies, this ΔCₜ when calculated as C_{t(T7 or T14)}-C_{t(T0)} accounts for the change in the stability of different analytes in unpreserved and preserved samples after storage at room temperature for a specified amount of time. ΔCₜ when calculated as C_{t(T0 Chem)}-C_{t(T0 NA)} accounts for the neutrality (change in the basal concentration of analytes with the addition of a given chemistry in the urine samples relative to the unpreserved urine samples at the time of collection, i.e. Day 0). Unchanged ΔCₜ values or ΔCₜ values close to 0 are indicative of stability, as this means that the concentration of analyte is not significantly changing over the course of time (and thus is indicative of the stability of the analyte in the composition under the testing conditions). For example, in the present cell-free DNA studies, ΔCₜ value ranged from +2 to +14 in unpreserved samples held at RT for 7 days. This marked increase in ΔCₜ value (median value: >+5) is indicative of degradation of cell-free DNA in unpreserved samples. On the other hand, ΔCₜ median value for the detection of cell-free DNA after storage at room temperature in the present aqueous stabilizing composition was almost zero, indicating preservation of cell-free DNA stability and content and also indirectly accounts for cellular stability and integrity. For cell-free RNA, median ΔCₜ value of +2.5 in unpreserved samples is indicative of cell-free RNA degradation, while relatively lower median ΔCₜ value of 1.3 is indicative of better stability of cell-free RNA content in preserved samples when compared to unpreserved samples. For cellular RNA stability, a median ΔCₜ value of +7.0 in unpreserved samples is indicative of marked degradation of cellular RNA. On the other hand, median ΔCₜ value of less than 2 in preserved samples indicate cellular RNA stability. Similarly, for EV RNA, a median ΔCₜ value of more than +3 in unpreserved samples is indicative of instability and compromised detection of EV RNA, while a median ΔCₜ value of 0.5 in preserved samples indicates excellent EV RNA stability and detection. This is merely one exemplary method of assessing stabilization of cells, extracellular vesicles, nucleic acids, and/or microorganisms in bodily fluids, and other methods of assessing such stabilization are known to the skilled worker and/or are outlined in further detail in the Materials and Methods section and Examples described below.

As described in further detail in Example 7 below, preservative agents/compositions containing formalin/formaldehyde-based fixatives may be used to fix cells in biological samples or specimens and prevent leaking of cellular nucleic acids into the extracellular space. Such compositions may contain formaldehyde, or alternatively compounds capable of releasing an aldehyde, such as a formaldehyde releaser/formaldehyde donor/formaldehyde-releasing preservative which is a chemical compound that slowly releases formaldehyde. Notably, when compared to the DNA isolated from frozen tissues, formalin-fixed tissues exhibit a high frequency of non-reproducible sequence alteration (Srinivasan M, Sedmak D, Jewell S (2002) Effect of fixatives and tissue processing on the content and integrity of nucleic acids. Am J Pathol 161(6): 1961-1971). Formaldehyde, a principal ingredient of most commonly used fixatives, leads to the generation of DNA-protein and RNA-protein cross-linkages. Furthermore, the nucleic acids will fragment in situations where the fixative solution is not buffered. Both of the above provide challenges for PCR-based analyses (Gilbert M T P, Haselkorn T, Bunce M, Sanchez J J, Lucas S B, Jewell L D, Van Marck E, Worobey M (2007) The isolation of nucleic acids from fixed, paraffin-embedded tissues—Which methods are useful when? PLoS ONE 2(6): e537. Doi: 10.1371/journal.pone.0000537; Wong S Q, Li J, Tan A Y-C, Vedururu R, Pang J-M B, Do H, Ellul J, Doig K, Bell A, MacArthur G A, Fox S B, Thomas D M, Fellowes A, Parisot J P, Dobrovic A (2014) Sequence artifacts in a prospective series of formalin-fixed tumours tested for mutations in hotspot regions by massively parallel sequencing. BMC Medical Genomics 7:23. Doi: 10.1186/1755-8794-7-23). Specifically, this chemical damage to DNA reduces Taq DNA polymerase fidelity and PCR amplification efficiency (Sikorsky J A, Primerano D A, Fenger T W, Denvir J (2007) DNA damage reduces Taq DNA polymerase fidelity and PCR amplification efficiency. Biochem Biophys Res Commun 355(2): 431-437). Hence, formalin/formaldehyde-based fixatives are not ideal for molecular analyses. Thus, an advantage of the aqueous stabilizing composition and method for preserving a bodily fluid at ambient temperature as disclosed herein is that the compositions and methods of the present application do not require the use of formaldehyde, or compounds/components capable of releasing an aldehyde such as formaldehyde releasers, formaldehyde donors or formaldehyde-releasing preservatives.

### EXAMPLES

Materials and Methods

Cell-Free Nucleic Acids Extraction:

Cell-free nucleic acids extraction was performed using QiaAmp Circulating Nucleic Acid Extraction Kit (Qiagen; Cat. No. 55114) according to manufacturer's protocol. First morning, First Void (FMFV) human urine, random mid-day first void (FV) urine samples and saliva samples were centrifuged down at 3000 g-3800 g for 10-20 minutes at room temperature (RT) and the cleared supernatant (2-4 mL) was used for cell-free nucleic acids extraction. Extracted cell-free nucleic acids profile was assessed on 4200 Agilent Tapestation platform using HS D5000 tapes (Agilent, Cat. No. 5067-5592) and reagents (Agilent, Cat. No. 5067-5593) according to manufacturer's instructions.

Urinary Extracellular Vesicles (EV) RNA Extraction:

Urine EV RNA extraction was performed using exoRNeasy Maxi Kit (Qiagen; Cat. No. 77164) or Ultrafiltration. Urine Samples were precleared by centrifugation at 3000×g for 10 minutes at RT, followed by filtration of supernatant using 0.80 μm syringe filter (Sartorius® Minisart NML®, Cat. No. 16592, or Millipore® Millex®-AA, Cat. No. SLAA033SB) prior to EV isolation and >200 nucleotide (nt) long RNA extraction according to manufacturer's instructions (Supplemental Information: Purification of exosomal RNA, including miRNA, from urine using the exoRNeasy Serum/Plasma Midi/Maxi Kit). EVs and EV RNA isolation using Ultrafiltration was performed using AMICON Ultra-15 centrifugal units with Ultracel-100 regenerated cellulose membrane (Millipore-Sigma; Cat. No. UFC910024) as follows:

1. Empty Ultracel-100 15 mL columns were washed with 1×PBS pH 7.4 (Thermo fisher Scientific; Cat. No. 10010023) using centrifugation at 4000 g for 5 mins at room temperature (RT).

2. Precleared and filtered urine samples were concentrated using Ultracel-100 columns by performing centrifugation at 4000 g for 10 mins at RT and the resulting filtrate was discarded.

3. Ultracel-100 15 mL columns filter with retained concentrated urine were washed with 1×PBS pH 7.4 (Thermo fisher Scientific; Cat. No. 10010023) by centrifugation at 4000 g for 5 mins at RT.

3. 700 μL of QIAzol Lysis Reagent Qiagen; Cat. No. 79306) was added directly to the washed Ultracel-100 filter for the lysis of captured EVs for EV RNA extraction. The filter columns were transferred to new 50 mL Falcon tubes; vortexed for 10 sec, incubated at RT for 5 mins followed by centrifugation at 4000 g for 5 mins at RT.

4. The resulting filtrate and the reminiscent lysate retained on the filter was collected for EV RNA isolation. Add 100 μL of chloroform and vortex vigorously. Let stand for 2-5 minutes at RT.

5. Centrifuge at 12,000×g for 15 minutes at 4° C. Transfer ˜400 μL of aqueous phase to a new tube.

6. Add 400 μL (equal volume) of 70% ethanol and mix properly prior to transfer of the mixture to Qiagen RNeasy MinElute columns. Centrifuge at 8,000×g for 30 seconds at RT. Discard the filtrate

7. Add 700 μL of Buffer RWT (Qiagen) to the columns. Centrifuge at 8,000×g for 30 seconds at RT. Discard the filtrate.

8. Add 500 μL of Buffer RPE (Qiagen) to the columns. Centrifuge at 8,000×g for 30 seconds at RT. Discard the filtrate.

9. Add 500 μL of Buffer RPE (Qiagen) to the columns. Centrifuge at 8,000×g for 2 minutes at RT. Discard the filtrate and transfer the empty columns to new 2 mL collection tubes (Qiagen). Centrifuge the columns with lids open at maximum speed for 5 mins to dry the membrane.

10. Add 20 μL of RNase-free water to the center of the dried spin columns. Let the columns stand at RT for 1 mins followed by centrifugation at maximum speed for 1 min at RT.

11. Store the collected RNA samples at −80° C. until quantification and downstream processing.

12. Extracted EV RNA samples were quantified on Agilent 2100 Bioanalyzer using Agilent RNA 6000 Pico Kit (Cat. No. 5067-1513) according to the manufacturer's instructions and/or Ribogreen quantification analysis using Quant-iT Ribogreen RNA Assay Kit (Thermo Fisher Scientific, Cat. No. R11490) for downstream cDNA preparations.

16S qPCR Assay:

Extracted nucleic acids from urine samples were subjected to qPCR assay for the quantification of bacterial DNA content using 2× iTaq Universal SYBR Mastermix (Bio-Rad; Cat. No. 1725121). The primers and qPCR conditions of the Bacterial 16s rRNA are as follows: BacrRNA173-Forward primer 5′ ATTACCGCGGCTGCTGG 3′ (SEQ ID NO: 1), BacrRNA173-Reverse primer 5′ CCTACGGGAGGCAGCAG 3′ (SEQ ID NO: 2) (D C Emery, D K Shoemark, T E Blatstone, C M Waterfall, J A Coghill, T A Cerajewska, M Davies, N X West, S J Allen (2017) 16S rRNA next generation sequencing analysis shows bacteria in Alzheimer's post-mortem brain. Frontiers in Aging Neuroscience 9: 195. Doi: 10.3389/friagi.2017.00195). The amplification mixture (20 μL) contained: 10 μL of 2×iTaq Universal SYBR mastermix, 1 μL each of 10 μM forward and reverse primer, 6 μL of nuclease-free water (NFW from Invitrogen, Cat. No. 10977023) and 2 μL of extracted urinary cell-free nucleic acids. E. coli gDNA standards with serial dilutions (1, 1:10, 1:100 and 1:1000) and a non-template control (2 μL of RNase/DNase-free water) were used in each qPCR run. PCR reactions were performed on a Bio-Rad C1000 Touch Thermal Cycler (#1851196) and conditions are as follows: 95° C.: 5 minutes, [95° C.: 20 seconds, 56° C.: 30 seconds]×45 cycles. Melt curves were obtained by heating the samples from 65° C. to 95° C. by increments of 0.5° C. and plate read for 5 seconds at every increment. Bacterial cell-free DNA or cellular DNA quantification analysis was performed using “ΔCₜ” which stands for [C_{t(T7)}-C_{t(T0)}]. “C_{t(T7)}” and “C_{t(T0)}” stands for qPCR cycle threshold at day 7 and day 0, respectively.

Human β-Globin qPCR Assay:

Extracted nucleic acids from urine samples were subjected to qPCR assay for the quantification of human cell-free DNA content using 2× iTaq Universal SYBR Mastermix (Bio-Rad; Cat. No. 1725121). The primers and the PCR conditions of the human β-globin qPCR assay are described in the literature (M Jung, S Klotzek, M Lewandowski, M Fleischhacker, K Jung (2003) Changes in concentration of DNA in serum and plasma during storage of blood samples. Clinical Chem 49(6): 1028-1029) and are as follows: Forward primer: 5′ ACACAACTGTGTTCACTAGC 3′ (SEQ ID NO: 3), reverse primer: 5′ CAACTTCATCCACGTTCACC 3′ (SEQ ID NO: 4). The amplification mixture (20 μL) contained: 10 μL of 2× iTaq Universal SYBR mastermix, 1 μL each of 10 μM forward and reverse primer, 6 μL of nuclease-free water (Invitrogen, Cat. No. 10977023) and 2 μL of extracted urinary cell-free nucleic acids. Human gDNA standards with serial dilution (1, 1:10, 1:100, 1:1000) and a non-template control (2 μL of RNase/DNase-free water) were used in each qPCR run. PCR reactions were performed on a Bio-Rad C1000 Touch Thermal Cycler (#1851196) and conditions are as follows: 95° C.: 5 minutes, [(95° C.: 20 seconds, 56° C.: 30 seconds)×45 cycles]. Melt curves were obtained by heating samples from 65° C. to 95° C. by increments of 0.5° C. and plate read for 5 seconds at every increment. For stability assessment: Human cell-free DNA quantification analysis was performed using “ΔCₜ” which stands for [C_{t(T)}-C_{t(T0)}]. “C_{t(T)}” stands for qPCR cycle threshold at day 7 or day 14, while “C_{t(T0)}” represents qPCR cycle threshold at day 0 for both the unpreserved and chemistry containing urine samples. Cell-free DNA quantification relative to unpreserved day 0 (NA) sample was quantified using ΔCₜ calculations as [C_{t(T)}-C_{t(T0 NA)}] where C_{t(T0 NA)} represents qPCR cycle threshold for day 0 unpreserved samples. Furthermore, to assess neutrality (i.e. change in the basal concentration of cell-free DNA with the addition of a given chemistry in the urine samples at the time of collection), ΔCₜ calculations were performed as [C_{t(T0 Chem)}-C_{t(T0 NA)}] where C_{t(T0 Chem)} represents qPCR cycle threshold for day 0 urine samples with chemistry/stabilization solution.

In-Vitro DNA Methylation Assay:

This assay was performed as described in the literature (C Ernst, P O McGowan, V Deleva, M J Meaney, M Szyf, G Turecki (2008) The effects of pH on DNA methylation state: In vitro and post-mortem brain studies. J Neurosci Methods 174(1):123-125). pGL3-basic plasmid (Promega; Cat. No. E1751) contains 25 CCGG sites. 1 μg of plasmid was treated with CpG methyl transferase (New England Biolabs; Cat. No. M0226S), an enzyme that methylates all cytosine nucleotides in a CpG dinucleotide according to the manufacturer's protocol. To confirm the methylation status, methylated plasmid (pGL3-CH3) was subjected to restriction endonuclease digestions with: HpaII and MspI. Both of these enzymes recognize the same site (CCGG). While HpaII is blocked from cutting DNA when the internal C is methylated; MspI is insensitive to the methylation status of the internal C. The in vitro-methylated pGL3 plasmid was column purified using Zymo Research's DNA Clean & Concentrator-5 kit (Cat. No. D4013). An equal amount of the purified plasmid was either spiked into 1× TE buffer pH 8.0 (positive control) or into male-pooled and female-pooled FMFV urine samples containing the composition of the present invention and the reaction tubes were kept at RT for 7 days. Following incubation, the DNA samples under went bisulfite conversion using Qiagen EpiTec Bisulfite Kit (Cat No. 59104). Bisulfite treatment will create a sequence difference between un-methylated plasmid (cytosines to uracil conversion) and methylated plasmid (methylated cytosines will remain immune to conversion) (Y Li and T O Tollefsbol (2011) DNA methylation detection: Bisulfite genomic sequencing analysis. Methods Mol Biol 791: 11-21. Doi: 10.1007/978-1-61779-316-5_2). PCR experiment using methylated plasmid-specific primers would generate a 278 bp amplicon. Primers were used as described in Ernst et al. (2008) supra (Forward primer: 5′-AAGATGTTTTTTTGTGATTGGT-3′ (SEQ ID NO: 5); Reverse primer: 5′-TTCCTATTTTTACTCACCCAAA-3′ (SEQ ID NO: 6)).

HPV Plasmid Spike-In Assay:

E. coli DH5a strain HPV16 plasmid (Human papilloma virus; type 16 clone) (ATCC Cat. No. 45113) was cultured in LB medium for the extraction of HPV16 plasmid using ZymoPURE II Plasmid Maxi prep Sample Kit (Zymo Research, Cat. Nos. D4202 & D4203). Extracted/purified plasmid was spiked in female-pooled and male-pooled first morning, first void urine samples at concentration (1-10 ng/mL), with and without the preservative chemistry of the present invention. A 200 μL aliquot of each plasmid-spiked urine sample was processed for total DNA extraction using QiaAmp DNA mini kit on QIAcube Connect. The amount of plasmid DNA in each reaction tube and at different days (T0 and T7) was quantified using a qPCR assay for the ampicillin resistance gene (Amp^{R}) found on the HPV16 plasmid backbone. The Amp^{R} qPCR primers and conditions are as follows: Forward Primer (FP): 5′AGCCATACCAAACGACGAG 3′ (SEQ ID NO: 7); Reverse primer (RP): 5′AGCAATAAACCAGCCAGCC 3′ (SEQ ID NO: 8). The amplification mixture (20 μL) contained 10 μL of 2× iTaq Universal SYBR mastermix, 1 μL each of 10 μM forward and reverse primer, 6 μL of nuclease-free water (Invitrogen, Cat. No. 10977023) and 2 μL of extracted urinary nucleic acids. HPV16 plasmid standards with serial dilution (1, 1:10, 1:100, 1:1000) and non-template control (2 μL of RNase/DNase-free water) was used in each qPCR run. PCR reactions were performed on a Bio-Rad C1000 Touch Thermal Cycler (#1851196) and the conditions are as follows: 95° C.: 5 minutes, [(95° C.: 20 seconds, 55° C.: 30 seconds)×45 cycles]. Melt curves were obtained by heating samples from 65° C. to 95° C. by increments of 0.5° C. and plate read for 5 seconds at every increment. HPV plasmid DNA quantification analysis was performed using “ΔCₜ” which stands for [C_{t(T7)}-C_{t(T0)}]. “C_{t(T7)}” and “C_{t(T0)}” stands for qPCR cycle threshold at day 7 and day 0, respectively.

Urinary Cell-Free and Cellular RNA Extraction:

Total cellular RNA from urine pellets was extracted using 1) Qiagen RNeasy plus Mini Kit (Cat. No. 74134) and eluted in 30 μL of RNase-free water according to manufacturer's instructions and/or 2) Trizol LS reagent (Sigma, Cat. No. T3934) as described below:

At each time point, samples were spun at 3800×g for 20 minutes. Pellets were resuspended in 750 μL of TRI Reagent LS (and 250 μL of water) at each time point. Samples were allowed to stand for 5 minutes before freezing at −80° C. The samples were thawed at RT and processed as follows:
1. Add 200 μL of chloroform and vortex vigorously. Let stand for 2-15 minutes at RT.
2. Centrifuge at 12,000×g for 15 minutes at 4° C. (volume of aqueous phase is about 70% of TRI Reagent volume). Transfer 500 μL of aqueous phase to a new tube.
3. Add 50 μL of 10× DNase buffer and 1 μL of RNase-free DNase (Lucigen, Cat. No. D9905K). Incubate at 37° C. for 15 minutes.
4. Add 500 μL (equal volume) of acid phenol chloroform and vortex vigorously. Let stand for 5 minutes followed by centrifuge at 12,000×g for 10 minutes at 4° C. Transfer the aqueous phase into a new tube and add 1 μL of 20 μg/μL of glycogen and 500 μL of isopropanol. Let stand for 10 minutes at RT.
5. Centrifuge at 12,000×g for 8 minutes at 4° C. Remove supernatant and wash pellet with 1 mL of 75% ethanol. Vortex sample and then centrifuge at 12,000×g for 5 minutes. Remove the supernatant and air dry pellet for 5-10 minutes.
6. Resuspend pellet in 30 μL of RNase-free water.

Total cell-free nucleic acids were extracted from supernatant using Qiagen Circulating Nucleic Acids Kit (Cat. No. 55114) and eluted in 30-50 μL of kit buffer AVE. RNA Profile Analysis was performed on 2100 Agilent Bioanalyzer using Pico6000 RNA assay (Cat. No. 5067-1513). mRNA Target Analysis was performed using Taqman based RT-qPCR assay for β-actin (ACTB: Hs00357333_g1) from Thermo Fisher Scientific (Cat. No. 4331182). For cell-free RNA quantification studies, prior to cDNA synthesis, cell-free DNA removal was performed using DNAse I digestion followed by RNA cleanup using RNeasy MinElute Cleanup Kit (Qiagen; Cat No. 74204) as per the instructions described in the QIAamp Circulating Nucleic Acids Kit (Qiagen; Cat. No. 55114).

RT-qPCR Assay for Cellular, Cell-Free and EV RNA:

cDNA was prepared with an equal amount (ng) of extracted RNA from each sample using random hexamers & M-MLV reverse transcriptase (Thermo Fisher Scientific; Cat. No. 28025-013), according to manufacturer's protocol; β-actin Taqman assay was performed with Taqman Gene Expression Master Mix II with UNG (Thermo Fisher Scientific; Cat. No. 4440038), according to manufacturer's protocol, using 2 μL of cDNA neat and each sample was run in either duplicate or triplicate. Initially, the efficiency of ACTB TaqMan assay was tested using serial dilutions of cDNA prepared from blood RNA. PCR reaction was performed in a Bio-Rad C1000 Touch Thermal Cycler (Cat. No. 1851196) and the conditions are as follows: 50° C.: 2 minutes, 95° C.: 10 minutes, [95° C.: 15 seconds, 60° C.: 1 minute]×45 cycles. RNA stability quantification was represented as “ΔCₜ” which stands for [C_{t(T7)}-C_{t(T0)}]. “C_{t(T7)}” and “C_{t(T0)}” stands for qPCR cycle threshold at day 7 and day 0, respectively. Furthermore, to assess neutrality (change in the basal concentration of analytes with the addition of a given chemistry in the urine samples at the time of collection), ΔCₜ calculations were performed as [C_{t(T0 Chem)}-C_{t(T0 NA)}] where C_{t(T0 Chem)} represents qPCR cycle threshold for day 0 urine samples with chemistry/stabilization solution.

Droplet Digital PCR (ddPCR) Analysis of DNA Samples for the Target β-Globin Gene:

Individual reactions for ddPCR contained a final primer concentration of 100 nM with 2× QX200 ddPCR EvaGreen Supermix (Bio-Rad; Cat. No. 1864034) in a final volume of 23 μL. 20 μL of the reaction mix was transferred a DG8 Cartridge (Bio-Rad; Cat. No. 1864008) with 65 μL of Droplet Generation Oil for EvaGreen (Bio-Rad; Cat. No. 1864006), covered with a DG8 Gasket (Bio-Rad; Cat. No. 1863009) and converted to droplets with the Bio-Rad QX200 Droplet Generator. Droplets were then transferred to a 96-well plate (Bio-Rad; Cat. No. 12001925) and heat sealed at 180° C. for 6 seconds with Pierce-able Foil Heat Seal (Bio-Rad; Cat. No. 1814040) using the Bio-Rad PX1 PCR Plate Sealer (Cat. No. 1814000). The samples were then cycled in a Bio-Rad C1000 Touch Thermal Cycler (Cat. No. 1851196) using a 3-step cycling program: 95° C. for 5 minutes, followed by 50 cycles of 95° C. for 30 seconds, annealing temperature set at 58° C. for 1 minute and 72° C. for 30 seconds, followed by 1 cycle each of 4° C. for 5 minutes, 90° C. for 5 minutes and hold at 12° C. The primers used in the β-globin ddPCR assay were same as used in the above mentioned the β-globin qPCR assay (Forward primer: 5′ ACACAACTGTGTTCACTAGC 3′ (SEQ ID NO: 3), reverse primer: 5′ CAACTTCATCCACGTTCACC 3′ (SEQ ID NO: 4)). All ramp rates were set at 2° C./second. The cycled plate was then transferred and read on the QX200 Droplet Reader (Bio-Rad; Cat. No. 1864003); data was analyzed with the Quanta-Soft Software (Bio-Rad; Cat. No. 1864011). For the analysis, the abundance was reported as concentration (copy number per μL) and the total accepted droplets were more than 10,000 droplets for a given sample.

Urinary Cellular DNA Extraction and Quantification:

Total cellular DNA from urine pellets was extracted using QiaAmp DNA mini kit (Qiagen; Cat. No. 51306) according to manufacturer's instructions and eluted in 50 μL of elution buffer or nuclease-free water (NFW). At each time point, samples were spun at 3800×g for 20 minutes. Urine pellets were kept frozen at −80° C. until extraction. Pellets were thawed at RT and resuspended in 200 μL of 1×PBS followed by total DNA extraction. Total cellular DNA quantification was performed using Quant-iT™ Picogreen™ dsDNA Reagent (Thermo Fisher Scientific; Cat. No. P7581). Total genomic DNA profile was assessed on Agilent 4200 Tapestation using Genomic DNA Tape according to the instructions. Targeted amplification of human genomic DNA was performed using GAPDH PCR for ˜1 Kb amplicon product. The primers and the PCR conditions of the GAPDH qPCR assay are as follows: Forward Primer: 5′-GTC AAC GGA TTT GGT CGT ATT G-3′ (SEQ ID NO: 9); Reverse Primer: 5′-CTC TCT TCC TCT TGT GCT CTT G-3′ (SEQ ID NO: 10). 95° C., 5 minutes, [95° C., 30 seconds; 56° C., 30 seconds; 72° C., 60 seconds]×25 cycles; 72° C., 10 minutes 4° C., hold. Each reaction was set up as follows:

| | | |
|---|---|---|
| | Final | μL Per |
| Reagent | Concentration | Reaction |

| | | |
|---|---|---|
| 10X PCR Buffer | 1X | 2.5 |

| | | | |
|---|---|---|---|
| 1 mg/mL BSA | 100 | μg/mL | 2.5 |
| 50 mM MgCl₂ | 3 | mM | 1.5 |
| 10 mM dNTPs | 0.3 | mM | 0.75 |
| 10 μM Forward Primer* | 0.5 | μM | 1.25 |
| 10 μM Reverse Primer* | 0.5 | μM | 1.25 |
| 5 U/μLTaq | 0.1 | U/μL | 0.5 |
| NFW | | | 12.75 |

| | | |
|---|---|---|
| Template DNA | — | (2) |

| | |
|---|---|
| Total | 25 |

In the Examples below, percentages of sugar in the compositions are in wt/vol, percentages of alkanol (e.g. methanol or ethanol) in the compositions are in vol/vol, and percentages of boric acid are in wt/vol.

### Example 1—Urinary Cell-Free DNA Content is Sample- and Sex-Dependent

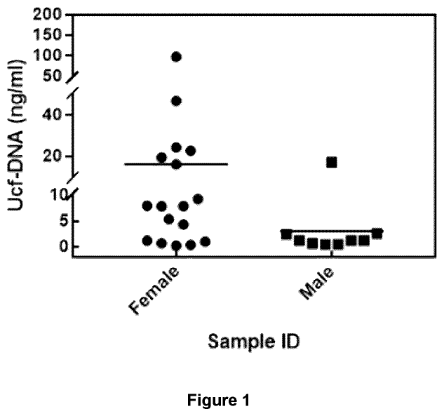

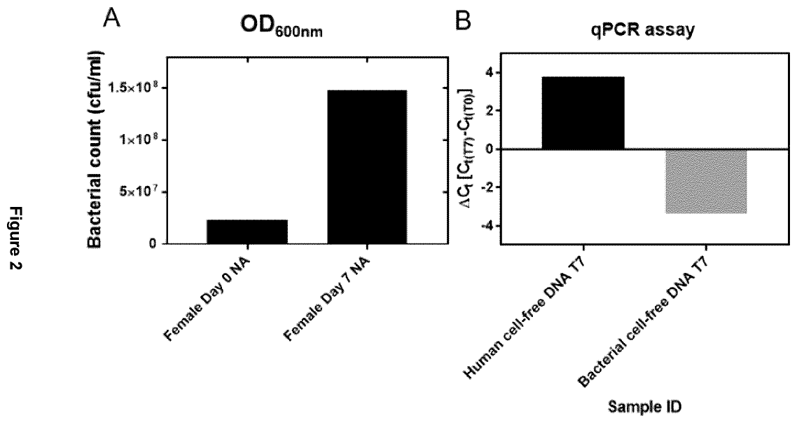

Approximately 20-30 mL of first morning, first void (FMFV) urine was collected from healthy female and male donors into urine specimen cups; transported and stored on ice packs until downstream processing. Within 3 hours of urine collection, a 4.5 mL aliquot of each specimen was centrifuged at 3,800 g for 20 minutes at room temperature. Cell-free nucleic acids were extracted from each 4.0 mL of the resulting supernatant either immediately or from frozen supernatant aliquots stored at −80° C. using the QIAamp Circulating Nucleic Acids Kit (Qiagen; Catalogue No. 55114; see Materials and Methods). Subsequently, urinary cell-free DNA (Ucf-DNA) concentration was measured using a Pico-Green quantification assay. The average urinary cell-free DNA concentration for female donors was about 15 ng/mL, compared to approximately 3 ng/mL for males (see FIG. 1). The presence of higher amounts of cell-free DNA in female urine than in male urine has also been reported in the literature (Streleckiene G, Reid H M, Arnold N, Bauerschlag D, Forster M. Quantifying cell free DNA in urine: comparison between commercial kits, impact of gender and inter-individual variation. Biotechniques. 2018, 64(5):225-230).

### Example 2—Human Cell-Free DNA Degrades in Unstabilized Urine Stored at Room Temperature

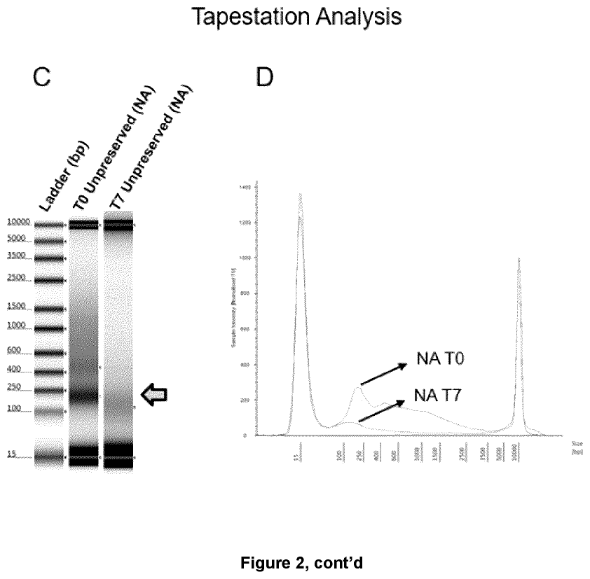

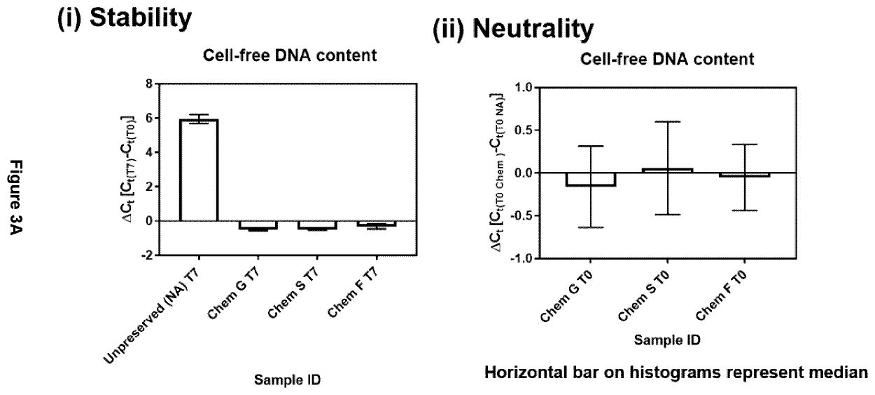

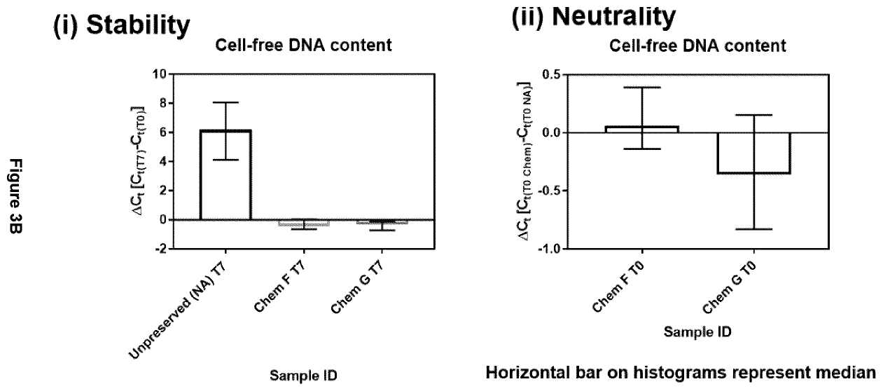

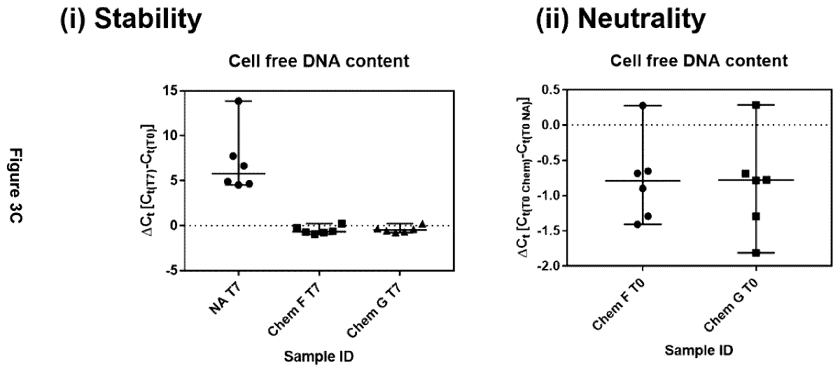

In the absence of a preservative or stabilizing agent (NA), urine stored at room temperature undergoes both visible and molecular changes. In this example, 20-30 mL of first morning, first void (FMFV) urine was collected from a healthy female donor and stored at room temperature for 7 days. During this period, this representative urine sample became increasingly turbid (FIG. 2A), as measured by an increase in bacterial count (OD_{600 nm}). This observation was further corroborated by quantitative bacterial 16S qPCR assay (FIG. 2B, see Materials and Methods), which showed a dramatic decrease in ΔCₜ for bacterial 16S DNA, demonstrating an increase in the bacterial cell-free DNA content due to the overgrowth and lysis of bacterial cells. In contrast, there was a dramatic increase in ΔCₜ for β-globin DNA demonstrating a significant decrease in human cell-free DNA content (FIG. 2B-D), as measured by a β-globin cell-free DNA qPCR assay (FIG. 2B; see Materials and Methods) and Agilent 4200 Tapestation analysis (see arrow in FIG. 2C-D, see Materials and Methods). Both methods, comparing cell-free DNA extracted from day zero and day 7 urine aliquots, clearly show a massive decline in cell-free DNA content after 7 days at room temperature (FIG. 2B-D).

### Example 3—Different Sugars (Monosaccharides/Disaccharides) can be Used in the Present Urine Stabilization Composition for Cell-Free DNA

Five healthy male and female donors provided a 60-70 mL first morning, first void (FMFV) urine specimen. Specimens were transported to the laboratory on ice packs where 1) 20 mL of each specimen was stored in the absence of a stabilizing composition (unpreserved), and 2) 12 mL of each urine specimen was mixed with 4 mL of stock solution [Table 1 (i)] containing different sugars, i.e. Glucose (Chem G), Sucrose (Chem S) and Fructose (Chem F), and 4 mL of 95% ethanol. In this example, final composition of the stabilization solution after mixing with urine is described below [see Table 1 (ii)]. Both types of specimens were stored at room temperature (23±3° C.) for at least 7 days.

On day zero and day 7, 4.5 mL aliquot of each unpreserved and different chemistries containing specimens were centrifuged at 3,800 g for 20 minutes at room temperature. 4.0 mL of supernatant was recovered from each specimen post-centrifugation and cell-free DNA was extracted using the QIAamp Circulating Nucleic Acids Kit (Qiagen, see Materials and Methods). Two microliters of purified cell-free DNA from each specimen served as template in β-globin qPCR analysis (see Material and Methods). FIGS. 3A and 3B illustrates a dramatic increase in ΔCₜ for β-globin DNA demonstrating a dramatic decrease in human cell-free DNA content after the unpreserved specimen was stored for 7 days at room temperature. In contrast, there was no significant change in human cell-free DNA levels in specimens with different sugars after 7 days at room temperature, as shown by ΔCₜ median value of almost zero [FIGS. 3A (i) and 3B (i)]. Moreover, there was no significant change in the cell free DNA content in the urine samples upon addition of the chemistries relative to unpreserved(NA) samples at the time of collection (day 0) [FIGS. 3A (ii) and 3B (ii)].

In another experimental setting, healthy male and female donors provided random first void (FV) urine specimen using Colli-Pee® device (Novosanis). Specimens were transported to the laboratory on ice packs where male and female urine samples were pooled to generate male pooled and female pooled specimens, respectively. An aliquot of each pooled specimen was stored 1) in the absence of a stabilizing composition (unpreserved), and 2) mixed with chemistries containing different sugars [Table 2 (i)], i.e. Glucose (Chem G), and Fructose (Chem F) in the urine: chemistry ratio of 1:0.43 An this example, final composition of the stabilization solution after mixing with urine is described below [see Table 2 (ii)]. All specimens were stored at room temperature (23±3° C.) for at least 7 days.

On day zero and day 7, 2.5 mL aliquot of each unpreserved and different chemistries containing specimen were centrifuged at 3,000 g for 10 minutes at room temperature followed by filtration using 0.8 μm syringe filters (Sartorius® Minisart NML®, Cat. No. 16592, or Millipore® Millex®-AA, Cat. No. SLAA033SB). 2.0 mL of precleared supernatant was used for cell-free DNA (cfDNA) extraction using the QIAamp Circulating Nucleic Acids Kit (Qiagen, see Materials and Methods). FIG. 3C (i) illustrates a dramatic increase in ΔCₜ (median value: +5.8) for β-globin DNA demonstrating a dramatic decrease in human cell-free DNA content after the unpreserved specimen was stored for 7 days at room temperature. In contrast, there was no significant change in ΔCₜ for β-globin DNA levels suggesting no change in human cell-free DNA content in Chemistry F (Chem F) and Chemistry G (Chem G) containing specimens after 7 days at room temperature [FIG. 3C (i)]. Moreover, there was no significant change in the cell free DNA content in the urine samples upon addition of the chemistries relative to unpreserved(NA) samples at the time of collection (day 0) [FIG. 3C (ii)].

The present composition with the disaccharide sucrose is difficult to prepare due to very high viscosity of the solution leading to improper mixing of the components. High viscosity can further lead to improper addition of stabilizing solution to the specimen due to difficulties in mixing. Therefore, to avoid these basic complications in preparation and testing of stabilizing solutions, it was decided to focus on monosaccharide-containing compositions being effective, while still maintaining sufficient stabilization of cell-free DNA content (FIGS. 3B and 3C). Overall, due to workability of the samples, monosaccharides are preferred over disaccharides for the present invention.

TABLE 1i
Compositions of different stock solutions
prior to mixing with urine.

| | Chemistry G | Chemistry S | Chemistry F |
|---|---|---|---|
| Composition | (Glucose) | (Sucrose) | (Fructose) |
| Sodium acetate | 1750 mM | 1750 mM | 1750 mM |
| Boric acid | 5% | 5% | 5% |
| CDTA | 119 mM | 119 mM | 119 mM |
| Sugar | 45% | 45% | 45% |
| pH | 4.7-5.0 | 4.7-5.0 | 4.7-5.0 |

TABLE 1ii
Final compositions of stabilizing solution after mixing with urine.

| | Chemistry G | Chemistry S | Chemistry F |
|---|---|---|---|
| Composition | (Glucose) | (Sucrose) | (Fructose) |
| Sodium acetate | 350 mM | 350 mM | 350 mM |
| Boric acid | 1% | 1% | 1% |
| CDTA | 23.8 mM | 23.8 mM | 23.8 mM |
| Sugar | 9% | 9% | 9% |
| Ethanol | 19% | 19% | 19% |

TABLE 2i
Compositions of different stock solutions
prior to mixing with urine.

| | Chemistry G | Chemistry F |
|---|---|---|
| Composition | (Glucose) | (Fructose) |
| Sodium acetate | 750 mM | 750 mM |
| Boric acid | 2.2% | 2.2% |
| CDTA | 50 mM | 50 mM |
| Sugar | 20% | 20% |
| Ethanol | 23% | 23% |
| pH | 5.0-5.2 | 5.0-5.2 |

TABLE 2ii
Final compositions of stabilizing solution after mixing with urine.

| | Chemistry G | Chemistry F |
|---|---|---|
| Composition | (Glucose) | (Fructose) |
| Sodium acetate | 225 mM | 225 mM |
| Boric acid | 0.7% | 0.7% |
| CDTA | 15 mM | 15 mM |
| Sugar | 6% | 6% |
| Ethanol | 6.9% | 6.9% |

### Example 4: Presence of Sugar, Alcohol, Buffer and Lower pH Modulates the Stabilization Effect of the Present Composition

Six healthy female donors provided a 30 mL first morning, first void urine (FMFV) specimen and their urine samples were pooled together to generate two different pooled urine specimens; 1) 15 mL of each pooled specimen was stored in the absence of a stabilizing composition (NA), and 2) 11 mL of each pooled urine specimen was mixed with 3 mL of stock solution (with different iterations of the present composition; Table 3 below) and 1 mL of 95% ethanol/methanol as described in Table 4. The final composition after mixing with the pooled urine is described in Table 5 below. All specimens were stored at room temperature (23±3° C.) for at least 7 days. For comparison, 25 mL of pooled urine was mixed with 5 mL of Streck's urine fixative (reference composition), commercially known as “Cell-free DNA Urine Preserve” (Cat. No. 230216), and stored at room temperature for at least 7 days. This reference composition comprises the formaldehyde releasing agent imidazolidinyl urea, as well as K₃EDTA and glycine.

On day zero and day 7, 4.5 mL aliquot of each unpreserved and chemistry containing pooled specimen was centrifuged at 3,800 g for 20 minutes at room temperature. 4.0 mL of supernatant was recovered from each specimen post-centrifugation and stored at −80° C. To assess the stability of the cell-free DNA with and without stabilization composition, frozen supernatant from both unpreserved and different chemistries containing day 0 and day 7 urine samples were subjected to cell-free DNA extraction using the QIAamp Circulating Nucleic Acids Kit (Qiagen, see Materials and Methods). Two microliters of purified cell-free DNA from each specimen served as template in β-globin qPCR analysis (see Material and Methods).

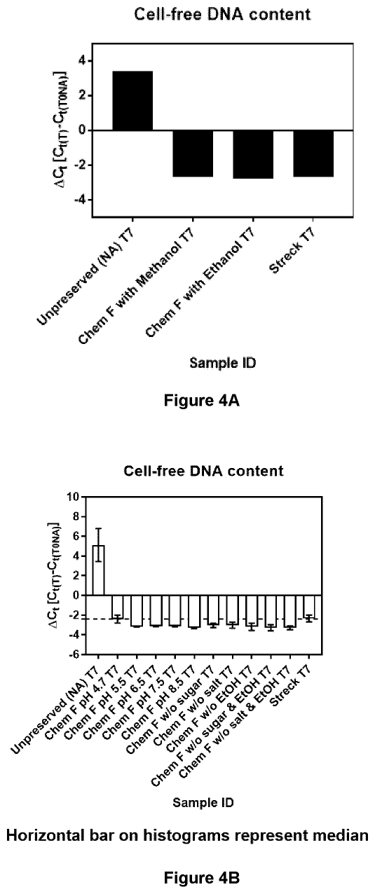

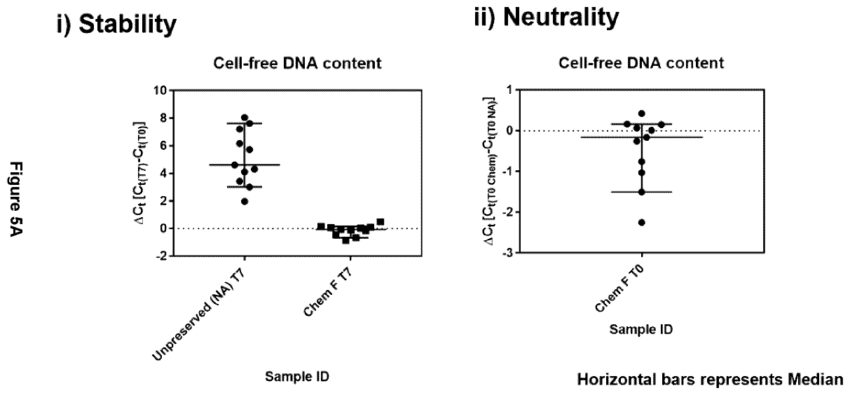

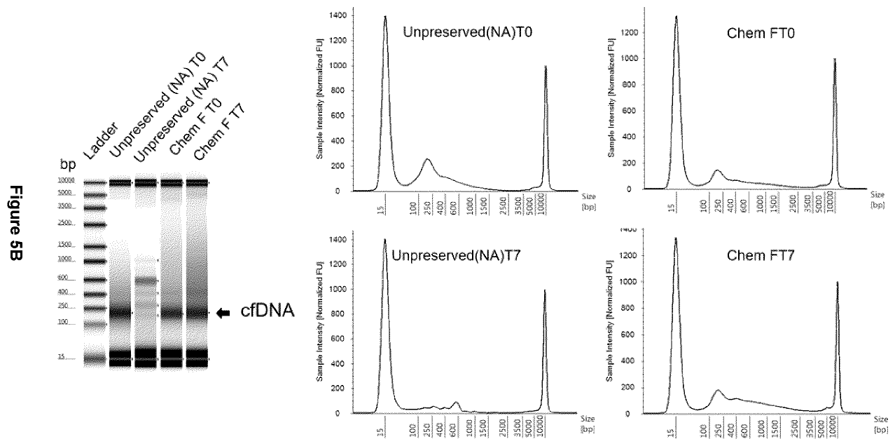

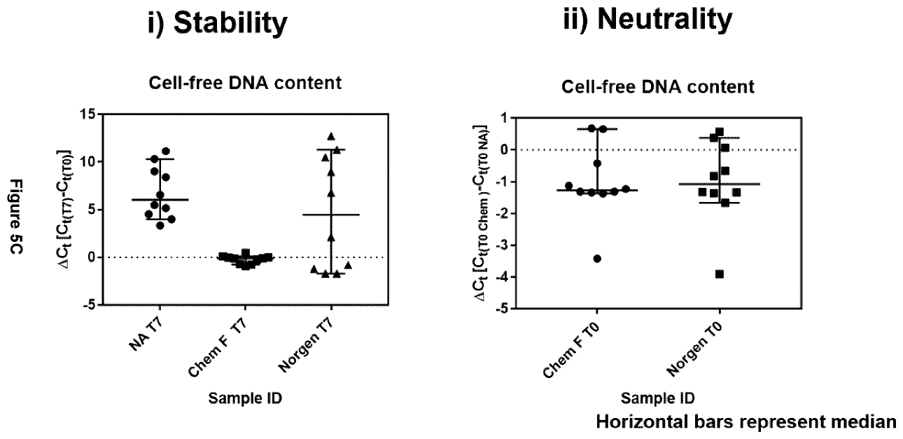

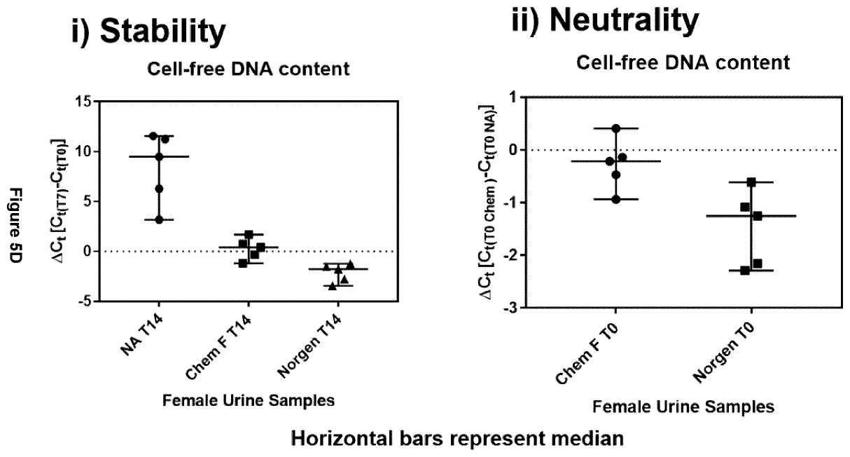

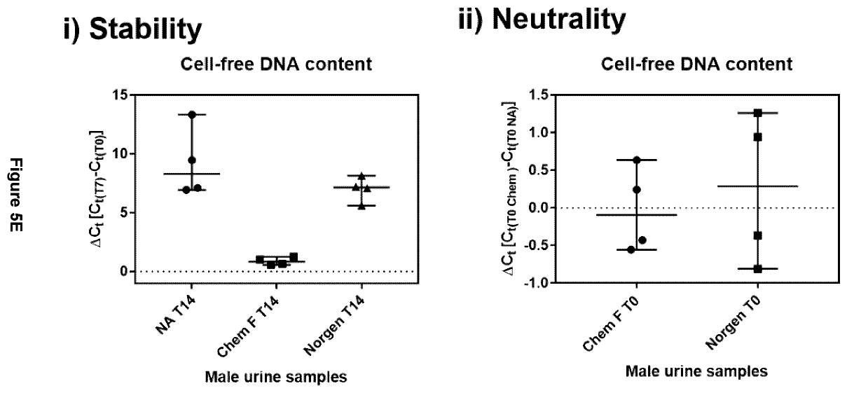

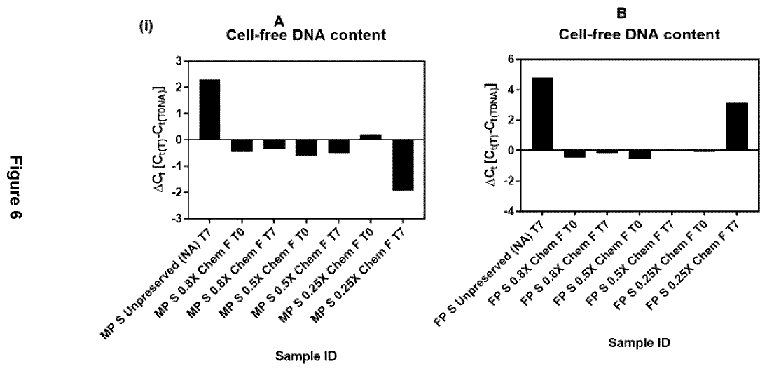

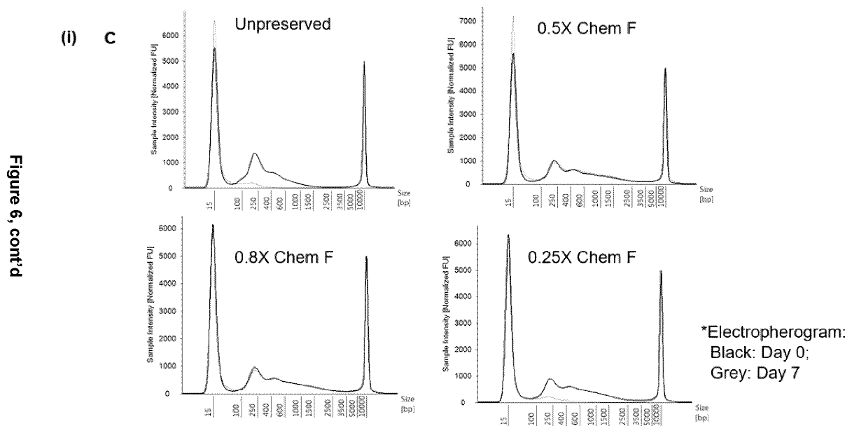

FIG. 4 (A&B) showed a dramatic increase in ΔCₜ for β-globin DNA demonstrating a dramatic decrease in human cell-free DNA content after the unpreserved specimen was stored for 7 days at room temperature (NA T7; FIGS. 4A & 4B). One of the pooled urine specimens was used to investigate the effect of different alcohols (ethanol and methanol) on the stabilization efficiency of the present composition. FIG. 4A suggests that ethanol in the present composition can also be substituted with methanol; however, methanol is toxic, at the concentrations used, and not ideal for at home collection, compared to ethanol. Moreover, human cell-free DNA in urine specimens with the present composition (Chem F, pH 4.7-5.0) was found to be similar to the Streck's urine fixative known as “Cell-free DNA Urine Preserve” after 7 days at room temperature (FIG. 4B), suggesting that the present composition is as effective as this reference composition as shown by similar ΔCₜ values for both the present and reference composition. Removal of either ethanol, buffer salt (e.g. sodium acetate), sugar, ethanol plus sugar, and ethanol plus salt, as well as elevated pH 5.5) made the Chemistry F composition less effective in preserving cell-free DNA content as indicated by a decrease in ΔCₜ values. This decrease in ΔCₜ suggests an increase in cell-free DNA content in the urine samples kept for 7 days at room temperature in different chemistry iterations, when compared to the complete composition of Chemistry F with ethanol (pH 4.7-5.0) (FIG. 4B). Finally, the data indicates that the ideal pH range for the present composition is 4.7-5.0 (+/−0.2).

TABLE 3
Composition of stock solutions prior to the mixture with urine.

| | | Chem F w/o | Chem F w/o |
|---|---|---|---|
| Composition | Chem F | Fructose | Buffer |
| Sodium acetate | 1750 mM | 1750 mM | — |
| Boric acid | 5% | 5% | 5% |
| CDTA | 119 mM | 119 mM | 119 mM |
| Fructose | 45% | | 45% |
| pH | 4.7-8.5 | 5.0 | 5.0 |

TABLE 4
Addition of different iterations in the urine sample.

| | Urine | Stock Solution | 95% Ethanol/Methanol |
|---|---|---|---|
| | (mL) | (mL) | (mL) |
| Chem F (pH 4.7-8.5) | 11 | 3 | 1 |
| Chem F w/o Fructose | 11 | 3 | 1 |
| Chem F w/o Buffer | 11 | 3 | 1 |
| Chem F w/o Ethanol | 12 | 3 | — |
| Chem F w/o Fructose | 12 | 3 | — |
| plus Ethanol | | | |
| Chem F w/o Buffer | 12 | 3 | — |
| plus Ethanol | | | |
| Chem F with Methanol | 11 | 3 | 1 |

TABLE 5
Final Composition after mixing with urine.

| | | Chem F w/o | Chem F w/o |
|---|---|---|---|
| Composition | Chem F | Fructose | Buffer |
| Sodium acetate | 350 mM | 350 mM | — |
| Boric acid | 1% | 1% | 1% |
| CDTA | 23.8 mM | 23.8 mM | 23.8 mM |
| Fructose | 9% | — | 9% |

### Example 5: Stabilizing Composition for the Preservation of Nucleic Acids in Urine at Room Temperature

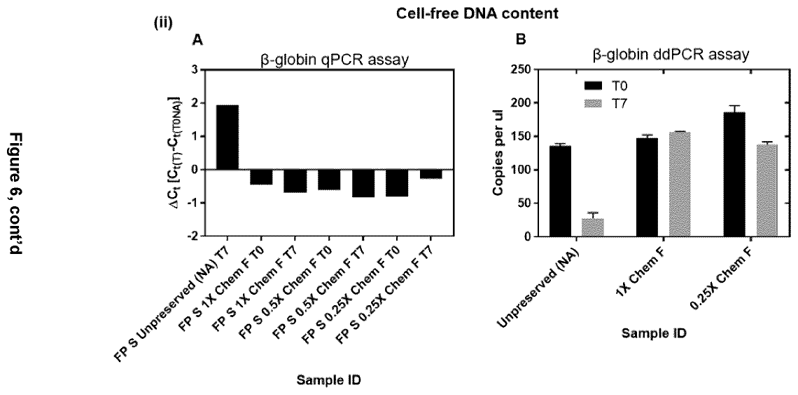

A total of eleven healthy donors (male and female) provided a 40-60 mL first morning, first void (FMFV) urine specimen. Specimens were transported to the laboratory on ice packs where i) 20 mL of each specimen was stored in the absence of a stabilizing composition (unpreserved), and 2) 12 mL of each urine specimen was mixed with stabilization solution [4 mL of stock solution; Table 6 (i), and 4 mL of 95% ethanol]. In this example, final composition of the stabilization solution after mixing with urine is described below [see Table 6 (ii)]. Both types of specimens were stored at room temperature (23±3° C.) for at least 7 days. On day zero and day 7, 4.5 mL aliquot of each unpreserved and specimen with stabilization solution was centrifuged at 3,800 g for 20 minutes at room temperature. 4.0 mL of supernatant was recovered from each specimen post-centrifugation and cell-free DNA (cfDNA) was extracted using the QIAamp Circulating Nucleic Acids Kit (Qiagen, see Materials and Methods). Two microliters of purified cfDNA from each specimen served as template in β-globin qPCR analysis (see Material and Methods). FIG. 5A illustrates a dramatic increase in ΔCₜ for β-globin DNA demonstrating a dramatic decrease in human cell-free DNA content after the unpreserved specimen was stored for 7 days at room temperature (FIG. 5A). In contrast, there was no significant change in ΔCₜ for β-globin DNA levels suggesting no change in human cell-free DNA levels in Chem F containing specimens after 7 days at room temperature [FIG. 5A (i)]. Moreover, there was no significant change in the cell free DNA content in the urine samples upon addition of the chemistries relative to unpreserved (NA) samples at the time of collection (day 0) [FIG. 5A (ii)]. Representative Tapestation profile analysis (FIG. 5B) using HSD5000 tape (Agilent Technologies) showed the presence of cell-free nucleic acids in unpreserved day zero aliquots, Chemistry F (Chem F) day zero and day 7 aliquots; cell-free nucleic acids were degraded in unpreserved day 7 aliquots.

In another experimental setting, urine samples from both male and female healthy donors were pooled to generate male and female pooled urine specimens. An aliquot of each specimen was stored in the 1) absence of a stabilizing composition (unpreserved), 2) mixed with the stock solution [Table 7 (i)] in 1:0.43 ratio and 3) mixed with Norgen urine collection and preservation tubes (Cat. 18111). In this example, final composition of the stabilization solution “Chemistry F (Chem F)” after mixing with urine is described below [see Table 7 (ii)]. All specimens were stored at room temperature (23±3° C.) for at least 7 days. On day zero and day 7, 2.5 mL aliquot of each unpreserved and stabilization solution containing urine specimen was centrifuged at 3,000 g for 10 minutes at room temperature followed by filtration using 0.8 μm syringe filters (Sartorius® Minisart NML®, Cat. No. 16592, or Millipore® Millex®-AA, Cat. No. SLAA033SB). 2.0 mL of supernatant was recovered from each specimen post-centrifugation and cell-free DNA (cfDNA) was extracted using the QIAamp Circulating Nucleic Acids Kit (Qiagen, see Materials and Methods). FIG. 5C (i) illustrates a dramatic increase in ΔCₜ for β-globin DNA demonstrating a dramatic decrease in human cell-free DNA content after the unpreserved specimen was stored for 7 days at room temperature [FIG. 5C (i)]. In contrast, there was no significant change in ΔCₜ for β-globin DNA levels suggesting no change in human cell-free DNA levels in Chemistry F (Chem F) containing specimens after 7 days at room temperature [FIG. 5C (i)]. On the other hand, samples containing Norgen urine preservative showed a marked increase in ΔCₜ for β-globin DNA demonstrating a dramatic decrease in human cell-free DNA content after the specimens were stored for 7 days at room temperature [FIG. 5C (i)]. Moreover, there was no significant change in the cell free DNA content in the urine samples upon addition of the chemistries relative to unpreserved(NA) samples at the time of collection (day 0) [FIG. 5C (ii)].

In another experimental setting, first void urine samples from healthy male and female donors collected using Colli-Pee® device (Novosanis) were pooled to generate male and female pooled urine specimens, respectively. An aliquot of each specimen was stored in the 1) absence of a stabilizing composition (unpreserved), 2) mixed with the stock solution (Table 7i) in 1:0.43 ratio and 3) mixed with Norgen urine collection and preservation tubes (Norgen Biotek; Cat. 18111). In this example, final composition of the stabilization solution after mixing with urine is described below (see Table 7ii). All specimens were stored at room temperature (23±3° C.) for at least 14 days. On day zero and day 14, 2.5 mL aliquot of each unpreserved and stabilization solution containing urine specimen was centrifuged at 3,000 g for 10 minutes at room temperature followed by filtration using 0.8 μm syringe filters (Sartorius® Minisart NML®, Cat. No. 16592, or Millipore® Millex®-AA, Cat. No. SLAA033SB). 2.0 mL of supernatant was recovered from each specimen post-centrifugation and cell-free DNA (cfDNA) was extracted using the QIAamp Circulating Nucleic Acids Kit (Qiagen, see Materials and Methods). FIGS. 5D (i) and 5E (i) illustrates a dramatic increase in ΔCₜ for β-globin DNA demonstrating a dramatic decrease in human cell-free DNA content after the unpreserved specimen was stored for 14 days at room temperature. In contrast, there was no significant change in ΔCₜ for β-globin DNA levels suggesting no change in human cell-free DNA levels in Chem F containing specimens after 14 days at room temperature [FIGS. 5D(i), 5E(i)]. On the other hand, samples containing Norgen urine preservative showed either a decrease in ΔCₜ for β-globin [FIG. 5D (i)] or an increase in ΔCₜ for β-globin DNA [FIG. 5E (i)] demonstrating an increase or decrease in human cell-free DNA content in the female and male urine specimens, respectively when stored for 14 days at room temperature. Moreover, Norgen urine preservative containing female urine samples showed changes in the neutrality when compared to Chem F samples at the time of addition (Day 0) [FIG. 5D (ii)].

TABLE 6i
Composition of stock solution prior to mixing with urine.

| Composition | Stock solution |
|---|---|
| Sodium acetate | 1750 mM |
| Boric acid | 5% |
| CDTA | 119 mM |
| Fructose | 45% |
| pH | 4.7-5.0 |

TABLE 6ii
Final compositions of stabilizing solution after mixing with urine.

| Composition | Stabilizing solution (Chem F) |
|---|---|
| Sodium acetate | 350 mM |
| Boric acid | 1% |
| CDTA | 23.8 mM |
| Fructose | 9% |
| Ethanol | 19% |

TABLE 7i
Composition of stock solution prior to mixing with urine.

| Composition | Stock solution |
|---|---|
| Sodium acetate | 750 mM |
| Boric acid | 2.2% |
| CDTA | 50 mM |
| Fructose | 20% |
| Ethanol | 23% |
| pH | 5.0-5.2 |

TABLE 7ii
Final compositions of stabilizing solution after mixing with urine.

| Composition | Stabilizing solution (Chem F) |
|---|---|
| Sodium acetate | 225 mM |
| Boric acid | 0.7% |
| CDTA | 15 mM |
| Fructose | 6% |
| Ethanol | 6.9% |

### Example 6: Stabilizing Composition Preserves the Integrity of Prostate Cancer Cells for 7 Days at Room Temperature

Urine from male donors may contain exfoliated prostate epithelial cells as a result of shedding from the prostate gland during normal turnover. Moreover, this secretion into urine can also be increased by physical manipulation of prostate gland by performing prostatic massage, especially in prostate cancer patients. Hence, to test the stability and intactness of cells in the stabilization solution containing urine sample, prostate cancer cells were used as one of the cell types of interest.

Cell-free DNA content over time was used to measure cellular integrity in the presence of the stabilizing composition, Chemistry F. In one experimental setting [Example 6(i)], first morning, first void urine (FMFV) specimens were pooled from 3 healthy male and 3 female donors to generate one female-pooled (FP) and one male-pooled (MP) urine specimen. Alongside male urine, female urine samples were also included in this study to test the stability of cancer cells in more concentrated, high biomass-containing urine matrix. The pooled specimens were centrifuged at 3,000 g for 10-20 minutes at room temperature, followed by filtration of the resulting supernatant using a 0.2 micron filter. These precleared, cell-free urine specimens were aliquoted and then spiked (S) with prostate cancer cells (LNCaP clone FGC; ATCC CRL-1740™).

To test the concentration-dependent effect of Chemistry F on the stability of spiked prostate cancer cells, varying amounts (mL) of stock solution (see Table 8) and a fixed amount of 95% ethanol were added to achieve different final concentrations of various components in Chemistry F after mixing with precleared urine containing spiked prostate cancer cells (see Table 9). The stock solution and ethanol were mixed with precleared urine containing spiked prostate cancer cells as described in Table 10.

In another experimental setting [Example (6ii)], first morning, first void urine (FMFV) specimens were pooled from three healthy females to generate one female-pooled (FP) urine specimen. The pooled specimens were centrifuged at 3,000 g for 10-20 minutes at room temperature, followed by filtration of the resulting supernatant using a 0.2 micron filter. These precleared, cell-free urine specimens were aliquoted and then spiked (S) with prostate cancer cells (LNCaP clone FGC; ATCC CRL-1740™). In this experimental setting, the amount (mL) of 95% ethanol was also varied along with variations in the amount of stock solution (mL) (Table 8) to achieve different final concentrations of components in Chemistry F after mixing with precleared urine containing spiked prostate cancer cells as specified in Table 11. The stock solution and ethanol amounts were mixed with precleared urine containing spiked prostate cancer cells as described in Table 12.

In both the experimental settings, the specimens were incubated with the present compositions for 30-60 minutes (day 0) or 7 days prior to cell-free DNA extraction using QIAamp Circulating Nucleic Acids Kit (Qiagen, see Materials and Methods). Extracted human cell-free DNA was quantified using β-globin qPCR assay (FIG. 6(i) (A&B) and FIG. 6(ii) A; see Materials and Methods) and normalized to day 0 unpreserved urine (NA).

FIG. 6(i) suggests that spiked human prostate cells did not leak genomic DNA into the supernatant in the presence of Chemistry F with a relatively constant amount of ethanol, in a concentration dependent manner. No Chemistry F (NA) addition resulted in a significant increase in ΔCₜ thus demonstrating decrease of cell-free DNA content [FIG. 6(i) A&B] in both male- and female-pooled specimens, compared to no significant change in 0.5× and 0.8× Chemistry F. On the other hand, 0.25× concentration showed either a decrease in ΔCₜ (meaning increased cfDNA content due to the compromised cellular stability leading to genomic DNA leakage) in MP urine sample or increase in ΔCₜ (meaning decreased cfDNA content due to compromised chemical stability leading to more degradation of cfDNA) in FP urine sample. This difference could be urine matrix dependent. Due to the presence of high biomass in the female urine sample, diluted concentration of components at 0.25× strength failed to inhibit the degradation of cell-free DNA from urine DNases and hence the rate of degradation is faster than the rate of preservation causing an overall cfDNA content loss. On the other hand, as the male urine matrix contains low biomass, concentration of chemistry components at 0.25× strength can still inhibit cell-free DNA degradation from urine DNases and hence the rate of preservation is faster than the rate of degradation leading to an overall increase in the cell-free DNA content. Overall, in both the FP and MP urine samples, there is a concentration-dependent effect of Chemistry F on cell-free DNA profile and cellular stability. Representative tapestation profile analysis of FP specimen (FIG. 6(i)C) also showed dramatic differences in the cell-free nucleic acids profile in the absence of Chemistry F (NA) and 0.25× Chemistry F between day 0 (black trace in FIG. 6(i)C) and day 7 (grey trace in FIG. 6(i)C), when compared to 0.8× and 0.5× diluted Chemistry F (FIG. 6(i)C).

FIG. 6(ii) A also suggests that spiked prostate cancer cells did not leak genomic DNA into the supernatant in the presence of Chemistry F with varying amounts of ethanol, in a concentration-dependent manner with 1× being most effective and 0.25× being least effective. 0.25× Chemistry F resulted in an initial decrease in ΔCₜ meaning increased cfDNA content at day 0, followed by an increase in ΔCₜ, suggesting decrease in cell-free DNA content at day 7 (FIG. 6(ii)A). β-globin qPCR assay results (FIG. 6(ii)A) were further corroborated by β-globin droplet digital PCR assay (FIG. 6(ii)B) which also revealed that 1× Chemistry F solution preserved the number of copies of β-globin gene per unit volume, while 0.25× Chemistry F resulted in an initial increase at Day 0 followed by significant decrease in the number of copies of β-globin gene per unit volume after 7 days at room temperature in the spiked urine specimen (FIG. 6(ii)B). Overall, the data suggests that the composition of the present invention preserves the integrity of prostate cancer cells in a concentration-dependent manner at room temperature for at least 7 days.

TABLE 8
Composition of Stock Solution

| Composition | Stock solution |
|---|---|
| Sodium acetate | 2800 mM |
| Boric acid | 8% |
| CDTA | 190.4 mM |
| Fructose | 72% |
| pH | 4.7-5.0 |

TABLE 9
Final composition of 0.8×, 0.5× and 0.25× Chemistry F after
mixing with precleared urine spiked with prostate cancer cells.

| | 0.8× | 0.5× | 0.25× |
|---|---|---|---|
| Composition | Chemistry F | Chemistry F | Chemistry F |
| Sodium acetate | 280 mM | 175 mM | 87.5 mM |
| Boric acid | 0.8% | 0.5% | 0.25% |
| CDTA | 19 mM | 11.9 mM | 5.95 mM |
| Fructose | 7.2% | 4.5% | 2.25% |
| Ethanol | 13.2% | 13.7% | 14.1% |

TABLE 10
Amount of Stock Solution and ethanol added to the
precleared urine spiked with prostate cancer cells.

| Final conc. | | | | |
|---|---|---|---|---|
| after | Stock | 95% | Precleared Urine | Total volume |
| mixing | Solution | Ethanol | containing spiked | (Urine + |
| with urine | (mL) | (mL) | cells (mL) | Chemistry) (mL) |
| 0.8× | 1.4 | 2 | 11 | 14.4 |
| 0.5× | 0.9 | 2 | 11 | 13.9 |
| 0.25 × | 0.45 | 2 | 11 | 13.45 |

TABLE 11
Final composition of 1×, 0.5× and 0.25× Chemistry F after mixing
with precleared urine spiked with prostate cancer cells.

| | 1× | 0.5× | 0.25× |
|---|---|---|---|
| Composition | Chemistry F | Chemistry F | Chemistry F |
| Sodium acetate | 350 mM | 175 mM | 87.5 mM |
| Boric acid | 1% | 0.5% | 0.25% |
| CDTA | 23.8 mM | 11.9 mM | 5.95 mM |
| Fructose | 9% | 4.5% | 2.25% |
| Ethanol | 11.88% | 5.94% | 2.97% |

TABLE 12
Amount of Stock Solution and ethanol added to the
precleared urine spiked with prostate cancer cells.

| Final conc. | Stock | 95% | Precleared Urine | Total volume |
|---|---|---|---|---|
| after mixing | Solution | Ethanol | containing spiked | (Urine + |
| with urine | (mL) | (mL) | cells (mL) | Chemistry) (mL) |
| 1× | 1 | 1 | 6 | 8 |
| 0.5× | 0.5 | 0.5 | 7 | 8 |
| 0.25 × | 0.25 | 0.25 | 7.5 | 8 |

### Example 7: Composition of the Present Invention Maintains the Integrity of Nucleated White Blood Cells Spiked into Precleared Urine Specimens and Stored at Room Temperature for 7 Days

Since bodily fluids (e.g. blood and urine) of most healthy individuals ordinarily do not contain substantial amounts of cell-free nucleic acids, elevated amounts of cell-free nucleic acids are usually indicative of a health issue (or pregnancy). However, after a blood sample is collected from a patient, cell lysis begins and the nucleic acids from within the blood cells are mixed with the cell-free nucleic acids, making it difficult to isolate and distinguish cell-free nucleic acids. In addition, these cell-free nucleic acids are susceptible to nuclease-initiated degradation in vitro. Consequently, the disease indication capability of cell-free nucleic acids may be diminished, as their presence is no longer accurately ascertainable. Ideally, prevention of cell lysis and cell-free nucleic acid degradation within the biological sample would allow for the cell-free nucleic acids to be accurately measured and the presence of any disease risk to be detected.

Preservative agents may be used to fix cells in biological samples or specimens and prevent leaking of cellular nucleic acids into the extracellular space. After the cell-free nucleic acids have been isolated, they can be tested to identify the presence, absence or severity of disease states including, but not limited to, a multitude of cancers. Pathology collections around the world represent an archive of genetic material to study populations and diseases. However, for preservation purposes, large portions of these collections have been fixed in formalin/formaldehyde-containing solutions, a treatment that results in cross-linking of biomolecules. A formaldehyde releaser, formaldehyde donor or formaldehyde-releasing preservative is a chemical compound that slowly releases formaldehyde. Notably, when compared to the DNA isolated from frozen tissues, formalin-fixed tissues exhibit a high frequency of non-reproducible sequence alteration (Srinivasan M, Sedmak D, Jewell S (2002) Effect of fixatives and tissue processing on the content and integrity of nucleic acids. Am J Pathol 161(6): 1961-1971). Formaldehyde, a principal ingredient of most commonly used fixatives, leads to the generation of DNA-protein and RNA-protein cross-linkages. Furthermore, the nucleic acids will fragment in situations where the fixative solution is not buffered. Both of the above provide challenges for PCR-based analyses (Gilbert M T P, Haselkorn T, Bunce M, Sanchez J J, Lucas S B, Jewell L D, Van Marck E, Worobey M (2007) The isolation of nucleic acids from fixed, paraffin-embedded tissues—Which methods are useful when? PLoS ONE 2(6): e537. Doi: 10.1371/journal.pone.0000537; Wong S Q, Li J, Tan A Y-C, Vedururu R, Pang J-M B, Do H, Ellul J, Doig K, Bell A, MacArthur G A, Fox S B, Thomas D M, Fellowes A, Parisot J P, Dobrovic A (2014) Sequence artifacts in a prospective series of formalin-fixed tumours tested for mutations in hotspot regions by massively parallel sequencing. BMC Medical Genomics 7:23. Doi: 10.1186/1755-8794-7-23). Specifically, this chemical damage to DNA reduces Taq DNA polymerase fidelity and PCR amplification efficiency (Sikorsky J A, Primerano D A, Fenger T W, Denvir J (2007) DNA damage reduces Taq DNA polymerase fidelity and PCR amplification efficiency. Biochem Biophys Res Commun 355(2): 431-437). Hence, formalin/formaldehyde-based fixatives are not ideal for molecular analyses.

In this example, the cellular stability of isolated white blood cells spiked into urine samples was assessed in the presence of the present preservative, compared to the formaldehyde-releasing preservative in Streck's Cell-Free DNA Urine Preserve (as described in Example 4). White blood cells were prepared from 1 mL of whole blood following selective lysis of red blood cells. The pelleted and washed white blood cells were spiked into urine samples and cfDNA content was used to measure the stability/intactness of the white blood cells. FMFV urine samples from female and male donors were pooled together to generate two female- and two male-pooled urine samples, respectively. The samples were “precleared” by centrifuging at 3,000 g for 10-20 minutes, followed by filtration of the supernatant using a 0.2-micron filter. The precleared urine samples were aliquoted and spiked with white blood cells followed by the addition of the present chemistry at a final concentration as mentioned in Table 13 (see below) or Streck's Cell-Free DNA Urine Preserve. The amount of stock solution (Table 14) and ethanol added to the precleared urine sample spiked with nucleated white blood cells is described in Table 15 (see below). Samples were incubated at room temperature for 30-60 minutes (day 0) or 7 days prior to cfDNA extraction using the QiaAmp Circulating Nucleic Acid Extraction Kit, according to the manufacturer's protocol. The extracted cfDNA was quantified using β-globin qPCR assay (see Materials and Methods).

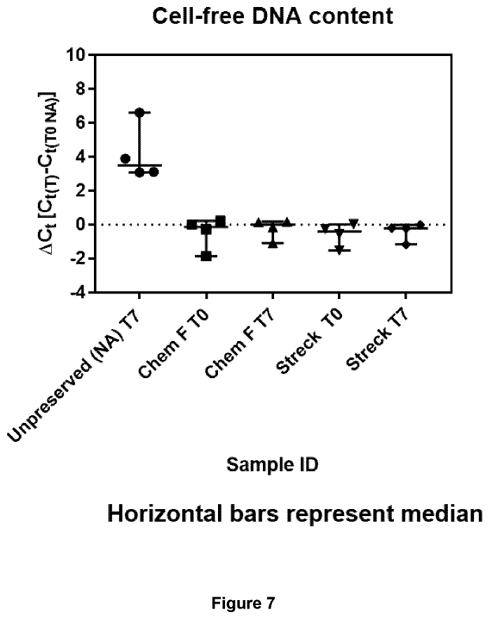

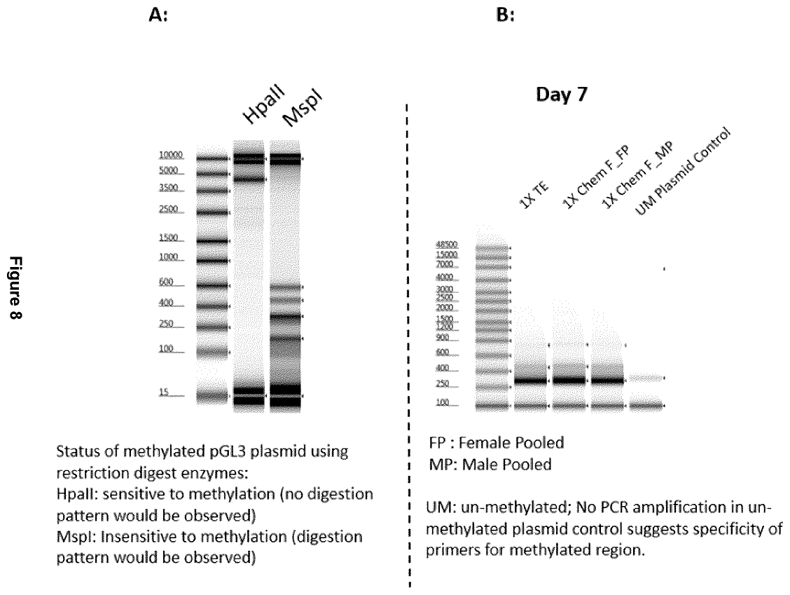

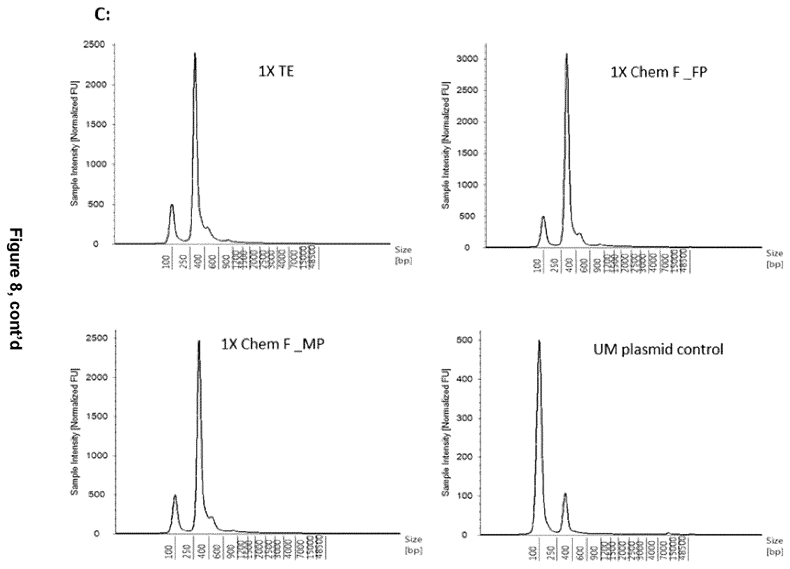

The data (see FIG. 7) suggests that the spiked white blood cells did not leak genomic DNA into the supernatant in the presence of the composition of the present invention, Chemistry F, after 7 days at room temperature with ΔCₜ median value of almost zero, suggesting preservation of cfDNA, as well as cellular stability and integrity over time. The composition of the present invention is functionally equivalent to Streck's formaldehyde-releasing chemistry in terms of stabilizing cfDNA at room temperature, without the risk of cross-linking DNA.

TABLE 13
Final concentration of the present composition after mixing
with urine spiked with nucleated white blood cells.

| | Stabilization |
|---|---|
| Composition | solution (Chem F) |
| Sodium acetate | 350 mM |
| Boric acid | 1% |
| CDTA | 23.8 mM |
| Fructose | 9% |
| Ethanol | 11.875% |

TABLE 14
Composition of Stock Solution

| Composition | Stock solution |
|---|---|
| Sodium acetate | 1750 mM |
| Boric acid | 5% |
| CDTA | 119 mM |
| Fructose | 45% |
| pH | 4.7-5.0 |

TABLE 15
Amount of Stock Solution and ethanol added to the precleared
urine spiked with nucleated white blood cells.

| Final conc. | Stock | 95% | Precleared Urine | Total volume |
|---|---|---|---|---|
| after mixing | Solution | Ethanol | containing spiked | (Urine + |
| with urine | (mL) | (mL) | cells (mL) | Chemistry) (mL) |
| 1× | 6 | 3.8 | 20.2 | 30 |

### Example 8: The Present Composition Preserves DNA Methylation Status for 7 Days at Room Temperature in Both Female-Pooled and Male-Pooled Urine Samples

DNA methylation, a process by which methyl groups are added to the DNA molecule, is one of several epigenetic mechanisms that cells use to control gene expression. It plays a pivotal role in many biological processes such as gene expression, embryonic development, cellular proliferation, differentiation and chromosome stability. Aberrant DNA methylation is often associated with the loss of DNA homeostasis and genomic instability leading to the development of diseases such as cancer (Y Li, T O Tollefsbol (2011) DNA methylation detection: Bisulfite genomic sequencing analysis. Methods Mol Biol 791: 11-21).

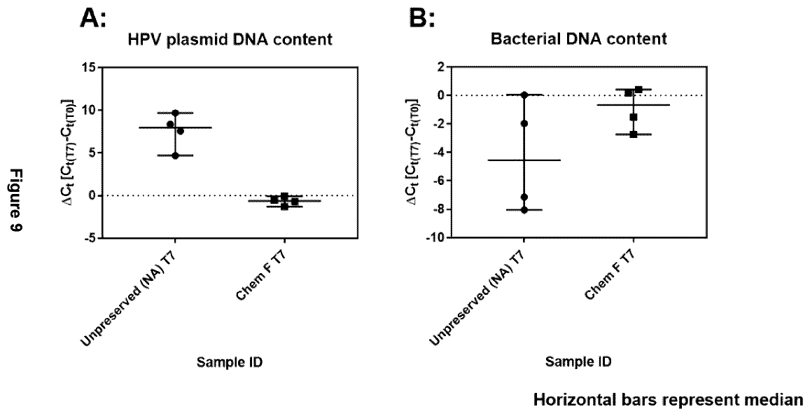

An ideal urine preservative solution must preserve the methylation status of DNA in studies involving DNA methylation as an epigenetic biomarker. Hence, to examine the effect of Chemistry F on DNA methylation status, an in vitro DNA methylation assay was performed using pGL3-basic plasmid which contains 25 CCGG sites. The assay involved the following steps as described in the Materials and Methods section. 1) In vitro methylation of plasmid followed by confirmation of methylation using restriction endonucleases digestion (FIG. 8A). 2) Bisulfite treatment of methylated plasmid incubated in control 1× TE buffer or in Chemistry F (1×) (see Table 16 below), followed by purification and PCR amplification of methylated plasmid using primers as described in the Materials and Methods section. The amount of stock solution (Table 17) and 95% ethanol added to the urine samples is described in Table 18. The presence of ˜278 base pair PCR product for methylated plasmid incubated in 1× TE buffer, as well as in Chemistry F solution (FIGS. 8B and 8C), suggests that methylation status of DNA in both female-pooled (FP) and male-pooled (MP) urine samples treated with Chemistry F was preserved for 7 days at room temperature.

TABLE 16
Final concentration of the composition after mixing with urine.

| Composition | Chemistry F |
|---|---|
| Sodium acetate | 350 mM |
| Boric acid | 1% |
| CDTA | 23.8 mM |
| Fructose | 9% |
| Ethanol | 19% |

TABLE 17
Stock Solution:

| Composition | Stock Solution |
|---|---|
| Sodium acetate | 1750 mM |
| Boric acid | 5% |
| CDTA | 119 mM |
| Fructose | 45% |
| pH | 4.7-5.0 |

TABLE 18

| Final concentration | Stock | 95% | Urine spiked with |
|---|---|---|---|
| after mixing with | Solution | ethanol | methylated plasmid |
| urine | (μL) | (μL) | (μL) |
| 1× Chem F | 10 | 10 | 30 |

### Example 9: The Composition of the Present Invention Preserves Human Papillomavirus (HPV) in First Morning, First Void Urine Samples after 7 Days Storage at Room Temperature

Cervical cancer is caused by sexually-acquired infection with certain types of genital HPV which are classified as high-risk and low-risk depending on their association with uterine cervical cancers (Munoz N, Bosch F X, de Sanjose S, Herrero R, Castellsaque X, Shah K V, Snijders P J, Meijer C J (2003) Epidemiologic classification of human papillomavirus types associated with cervical cancer. N Engl J Med 348(6): 518-527. Doi: 10.1056/NEJMoa021641). HPV16, 18, 31, 33, 35, 45, 52, 58, 39, 51, 56, and 59 have been classified as high risk HPV genotypes (Bouvard V, Baan R, Straif K, Grosse Y, Secretan B, E I Ghissassi F, Benbrahim-Tallaa L, Guha N, Freeman C, Galichet L, Cogliano V (2009) A review of human carcinogens—Part B: Biological agents. The Lancet Oncology 10: 321-322), out of which two HPV types (16 and 18) are the major cause (70%) of cervical cancers and pre-cancerous cervical lesions according to the WHO.

Urine being non-invasive provides a simple and feasible alternative to HPV detection in cervical specimens based on the literature around HPV detection (Vorsters, P Van Damme, G Clifford (2014) Urine testing for HPV: rationale for using first void. BMJ 349: g6252; Bernal, S. et al., Comparison of urine and cervical samples for detecting human papillomavirus (HPV) with the Cobas 4800 HPV test, Journal of Clinical Virology 61 (2014) 548-552; Enerly, E. et al., Monitoring human papillomavirus prevalence in urine samples: a review, Clinical Epidemiology 2013:5 67-79). In this study, the effect of the present composition on the stability of urine spiked with exogenous HPV circular DNA (HPV 16) has been evaluated. This system presents the most challenging scenario (non-protected circular DNA floating in the urine space/matrix) as compared to the mixed population of endogenous viral particles which would be present in both the protected (particles inside the cervical cells and/or covered with host proteins), as well as in non-protected state in HPV16 infected patient urine samples.

Healthy male and female donors provided first morning, first void (FMFV) urine specimens which were transported to the laboratory on ice packs and pooled together to generate two male- and two female-pooled urine samples. Purified HPV16 plasmid DNA (see Materials and Methods) was spiked into approximately 1 mL of pooled FMFV urine samples at a concentration of 1-10 ng/mL, with and without the composition of the present invention, Chemistry F (pH 4.7-5.0), and stored at room temperature for up to 7 days. The final concentration of the components in the stabilization composition “Chemistry F (Chem F)” after combining with the urine sample is described in Table 19 (below). On day 0 and day 7, a 200 μL aliquot of each of the HPV16 plasmid-spiked urine sample was processed for total DNA extraction using QiaAmp DNA mini kit according to the manufacturer's protocol. The DNA was eluted using 100 μL of kit elution buffer. The extracted DNA was further subjected to a qPCR assay for the HPV16 plasmid DNA quantification using Ampicillin resistance gene (Amp^{R}) on the HPV16 plasmid backbone. Bacterial DNA was quantified using 16S qPCR assay (see Materials and Methods).

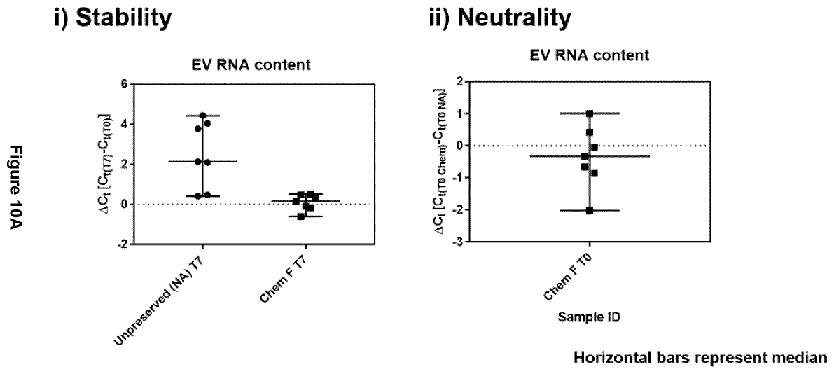

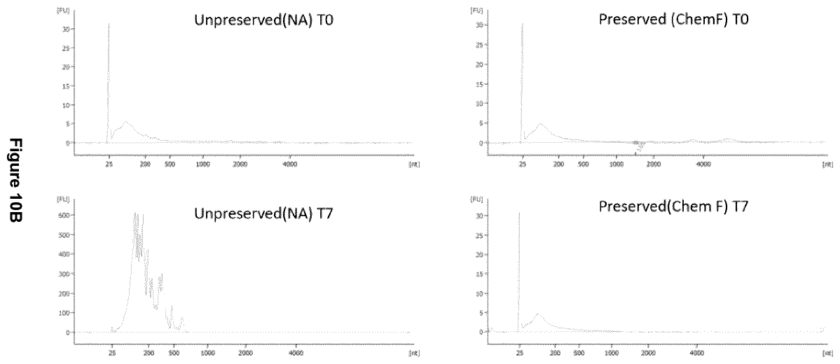

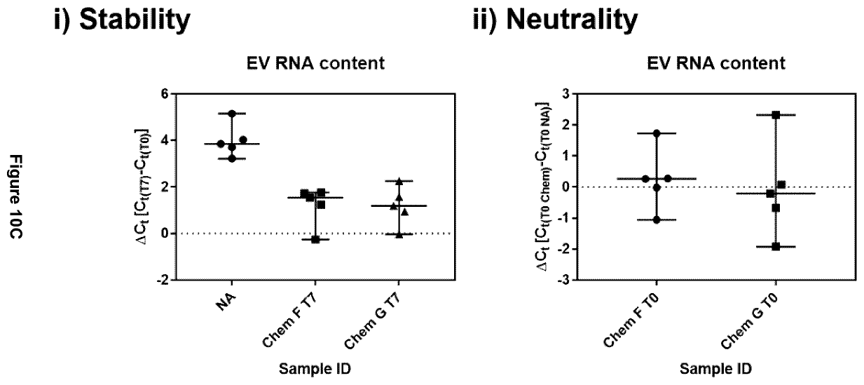

After 7 days at room temperature, the composition of the present invention stabilized exogenous spiked-in HPV16 plasmid DNA in FMFV urine samples as shown by ΔCₜ median value close to zero in preserved urine specimens, unlike in unpreserved specimens which showed marked increase in ΔCₜ median value (FIG. 9A). In addition, the composition of the present invention prevented an increase in bacterial DNA in FMFV urine samples as shown by ΔCₜ median value close to zero (FIG. 9B), unlike in unpreserved specimens which showed a marked decrease in ΔCₜ median value suggesting an increase in the bacterial DNA content after storage at RT for 7 days. The stability results obtained from spiked HPV16 DNA in urine samples can be extrapolated to the stability of endogenous HPV16 particles present in the patient samples.

TABLE 19
Final concentration of the stabilization composition
“Chem F” in the urine sample.

| | Stabilizing |
|---|---|
| Composition | Solution (Chem F) |
| Sodium acetate | 350 mM |
| Boric acid | 1% |
| CDTA | 23.8 mM |
| Fructose | 9% |
| Ethanol | 6% |

### Example 10: Stabilizing Composition for the Preservation of Extracellular Vesicles (EV) RNA in Urine at Room Temperature

Urine, being non-invasive as a sample type, has an obvious advantage over blood when used for liquid biopsy purposes. Urine contains prostate secretions and hence represents a potential valuable source for the detection and monitoring of prostate cancer. Prostate cancer is the second leading cause of cancer-related death in men and the most commonly diagnosed male malignancy worldwide, with >1.1 million cases recorded in 2012 (http://www.cancerresearchuk.org/) (O E Bryzgunova, M M Zaripov, T E Skvortsova, E A Lekchnov, A E Grigor'eva, I A Zaporozhchenko, E A Morozkin, E I Ryabchikova, Y B Yurchenko, V E Voitsitskiy, P P Laktionov (2016) Comparative study of extracellular vesicles from the urine of healthy individuals and prostate cancer patients. PLoS One 11(6): e0157566. Doi: 10.1371/joumal.pone.0157566).

The most well characterized urine biomarker for prostate cancer is a non-coding EV RNA known as PCA3 (DD3) with an increased expression in prostate cancer (M J Bussemakers, A van Bokhoven, G W Verhaegh, F P Smit, H F M Karthaus, J A Schalken, F M J Debruyne, N Ru, W B Isaacs (1999) DD3: a new prostate-specific gene, highly overexpressed in prostate cancer. Cancer Res 59: 5975-5979; K L Pellegrini, D Patil, K J S Douglas, G Lee, K Wehrmeyer, M Torlak, J Clark, C S Cooper, C S Moreno, M G Sanda (2018) Detection of prostate cancer-specific transcripts in extracellular vesicles isolated from post-DRE urine. Prostate 77(9): 990-999. Doi: 10.1002/pros.23355). ExoDx Prostate test (Exosome Diagnostics) is also based on urinary exosome RNA content for the prediction of high-grade prostate cancer (J McKiernan, M J Donovan, V O'Neill, S Bentink, M Noerholm, S Belzer, J Skog, M W Kattan, A Partin, G Andriole, G Brown, J T Wei, I M Thompson, P CVarroll (2016) A novel urine exosome gene expression assay to predict high-grade prostate cancer at initial biopsy. JAMA Oncol 2(7): 882-889. Doi: 10.1001/jamaoncol.2016.0097). The potential for microbial proliferation and the labile nature of host cells and extracellular vesicles (EVs) at the point of sample collection and transport to the lab drive the need for stabilization of urine samples for home sampling, as multi-site collections and at-clinic collections are increasingly prohibitive for large-scale recruitment and lead to variability in the time between collections and processing. Therefore, development of urine stabilization for home sampling opens up new applications for various urine derived biomarkers (e.g. urinary EV RNAs in prostate cancer) to be used in liquid biopsy analysis.

In one of the experimental settings, first morning first void urine samples were collected from healthy male and female donors in the standard urine collection cup. Specimens were transported to the laboratory on ice packs where samples were pooled together to form pooled urine specimens (MP, male-pooled; FP, female-pooled). i) 30 mL of pooled urine was stored in the absence of a stabilizing composition (unpreserved), and 2) 24 mL of pooled urine specimen was mixed with stabilization composition [4 mL of stock solution (Table 20) and 2 mL of 95% ethanol] and stored. The composition of the stock solution is described in Table 20. Both types of specimens were stored at room temperature (23±3° C.) for at least 7 days. The final composition of the stabilization solution “Chemistry F (Chem F)” after mixing with urine is described in Table 21. On day zero and day 7, 10 mL aliquot of each unpreserved and Chem F containing urine specimen was centrifuged at 3,000 g for 10 minutes at room temperature, followed by 0.8 μM filtration. Precleared supernatant recovered from each specimen post-centrifugation and filtration was used for EV RNA extraction with the ExoRNeasy maxi kit (Qiagen, see Materials and Methods). The concentration of extracted RNA samples was measured using 2100 Agilent Bioanalyzer and/or Ribogreen quantification. cDNA was prepared using the M-MLV Reverse Transcription kit and qPCR was performed using β-actin (ACTB) TaqMan assay (see Materials and Methods). For cDNA synthesis, an equal amount (ng) of total extracted RNA from the unpreserved and stabilized condition was used for a given urine sample.

In another experimental setting, male and female healthy donors provided random (mid-day), first void urine sample using the Colli-Pee® First Void Urine Collection Device (Novosanis). Specimens were transported to the laboratory on ice packs where samples were pooled together to form pooled urine specimens (MP, male-pooled; FP, female-pooled). i) 40 mL of pooled urine was stored in the absence of a stabilizing composition (unpreserved), and 2) 28 mL of pooled urine specimen was mixed with 12 mL of Chemistry F (Chem F) stabilizing composition and stored. The composition of the stabilization solution is described in Table 22 i. Both types of specimens were stored at room temperature (23±3° C.) for at least 7 days. The final composition of the stabilization solution “Chem F” after mixing with urine is described in Table 22 ii. On day zero and day 7, 17 mL aliquot of each unpreserved and Chem F containing specimen was centrifuged at 3,000 g for 10 minutes at room temperature, followed by 0.8 μM filtration. 16 mL of precleared supernatant was recovered from each specimen post-centrifugation and filtration and EV RNA was extracted using the ExoRNeasy maxi kit (Qiagen, see Materials and Methods). The concentration of extracted RNA samples was measured using 2100 Agilent Bioanalyzer and/or Ribogreen quantification (see Materials and Methods). The profile of the extracted EV RNAs was also determined on 2100 Agilent Bioanalyzer. For cDNA synthesis, an equal amount (ng) of total extracted RNA from the unpreserved and stabilization condition was used for a given urine sample. cDNA was prepared using the M-MLV Reverse Transcription kit and qPCR was performed using β-actin TaqMan assay (see Materials and Methods). β-actin has been referred to as a housekeeping gene for exosomal mRNA quantification using qPCR assay (H Jiang, Z Li, X Li, J Xia (2015) Intercellular transfer of messenger RNAs in multiorgan tumorigenesis by tumor cell-derived exosomes. Mol Med Rep 11: 4657-4663. Doi: 10.3892/mmr.2015.3312; K C Miranda, D T Bond, M McKee, J Skog, T G Paunescu, N Da Silva, D Brown, L M Russo (2010) Nucleic acids within urinary exosomes/microvesicles are potential biomarkers for renal disease. Kidney Int 78(2): 191-199. Doi: 10.1038/ki.2010.106; S Haque, S R Vaiselbuh (2018) Exosomes molecular diagnostics: direct conversion of exosomes into the cDNA for gene amplification by two-step polymerase chain reaction. J Biol Methods 5(3): e96. Doi: 10.14440/jbm.2018.249; L Dong, W Lin, P Qi, M Xu, Z Wu, S Ni, D Haung, W-W Weng, C Tan, W Sheng, X Zhou, X Du (2016) Circulating long RNAs in serum extracellular vesicles: their characterization and potential application as biomarkers for diagnosis of colorectal cancer. Cancer Epidemiol Biomarkers Prev 25(7):1158-1166. Doi: 10.1158/1055-9965. EPI-16-0006).

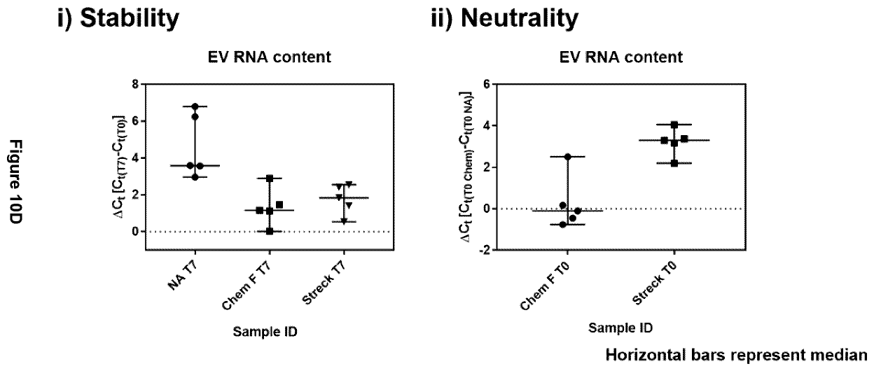

Overall data from the total of 7 samples (3 female-pooled and 4 male-pooled urine samples) from both experimental set-ups is combined and presented in FIG. 10A. FIG. 10A illustrates ΔCₜ which stands for [C_{t(T7)}-C_{t(T0)}] for β-actin (ACTB) RNA content in both unpreserved and stabilization solution containing urine specimens after storage for 7 days at RT. An increase in ΔCₜ (ΔCₜ median value of ≥+2, FIG. 10A) for β-actin (ACTB) RNA demonstrates loss of EV RNA content in unpreserved specimens stored for 7 days at room temperature. However, change in ΔCₜ for β-actin RNA in the Chem F containing urine specimens was insignificant (ΔCₜ median value of almost 0; FIG. 10A) demonstrating stabilization of EV RNA content after 7 days at room temperature. Moreover, there was no significant change in the EV RNA content in the urine samples upon addition of the chemistries relative to unpreserved(NA) samples at the time of collection (day 0) [FIG. 10A (ii)]. FIG. 10B illustrates representative electropherogram traces of EV RNA from both unpreserved and Chem F containing urine specimen at day 0 and day 7. Electropherogram traces clearly indicate marked change in the EV RNA profile in unpreserved urine specimen at day 7, unlike Chem F containing day 0 and day 7 specimens which showed EV RNA profile similar to unpreserved day 0 specimen.

In another experimental setting, male and female healthy donors provided random (mid-day), first void urine sample using the Colli-Pee® First Void Urine Collection Device (Novosanis). Specimens were transported to the laboratory on ice packs where samples were pooled together to form pooled urine specimens (MP, male-pooled; FP, female-pooled). An aliquot of pooled urine was stored in the 1) absence of a stabilizing composition (unpreserved), 2) mixed with stock solutions [Table 22 (iii)] containing different sugars in the urine: chemistry ratio of 1:0.43 and stored. All specimens were stored at room temperature (23±3° C.) for at least 7 days. The final composition of the stabilization solution after mixing with urine is described in Table 22 (iv). On day zero and day 7, 8.5 mL aliquot of each unpreserved, Chem F and Streck preservative containing urine specimen was centrifuged at 3,000 g for 10 minutes at room temperature followed by 0.8 μM filtration (Sartorius® Minisart NML®, Cat. No. 16592, or Millipore® Millex®-AA, Cat. No. SLAA033SB). 8 mL of precleared supernatant was recovered from each specimen post-centrifugation and filtration and EV RNA was extracted using ultrafiltration (see EV RNA extraction in Materials and Methods). The concentration of extracted RNA samples was measured using Ribogreen quantification (see Materials and Methods). For cDNA synthesis, an equal amount (ng) of total extracted RNA from the unpreserved and stabilization condition was used for a given urine sample. cDNA was prepared using the M-MLV Reverse Transcription kit and qPCR was performed using β-actin TaqMan assay (see Materials and Methods).

FIG. 10C (i) illustrates ΔCₜ which stands for [C_{t(T7)}-C_{t(T0)}] for β-actin (ACTB) RNA content in both unpreserved and stabilization solutions containing urine specimens after storage for 7 days at RT. An increase in ΔCₜ [ΔCₜ median value of >+3.5, FIG. 10C (i)] for β-actin (ACTB) RNA demonstrates loss of EV RNA content in unpreserved specimens stored for 7 days at room temperature. Chem F and Chem G containing specimens showed median ΔCₜ values of 1.5 and 1.1, respectively for β-actin RNA demonstrating efficient stabilization of EV RNA content after 7 days at room temperature [FIG. 10C (i)]. Moreover, there was no significant change in the EV RNA content in the urine samples upon addition of the chemistries relative to unpreserved(NA) samples at the time of collection (day 0) [FIG. 10C (ii)]

In another experimental setting, male and female healthy donors provided random (mid-day), first void urine sample using the Colli-Pee® First Void Urine Collection Device (Novosanis). Specimens were transported to the laboratory on ice packs where samples were pooled together to form pooled urine specimens (MP, male-pooled; FP, female-pooled). An aliquot of pooled urine was stored in the 1) absence of a stabilizing composition (NA, unpreserved), 2) mixed with Chemistry F stabilizing composition in the urine: chemistry ratio of 1:0.43 and 3) mixed with 5 mL of Streck's urine preservative (Cat. No. 230216) and stored. The composition of the stabilization solution is described in Table 23 (i). Both types of specimens were stored at room temperature (23±3° C.) for at least 7 days. The final composition of the stabilization solution after mixing with urine is described in Table 23 (ii). On day zero and day 7, 11 mL aliquot of each unpreserved, Chem F and Streck preservative containing urine specimen was centrifuged at 3,000 g for 10 minutes at room temperature, followed by 0.8 μM filtration. 10 mL of precleared supernatant was recovered from each specimen post-centrifugation and filtration and EV RNA was extracted using the ExoRNeasy Maxi kit (Qiagen, see Materials and Methods). The concentration of extracted RNA samples was measured using Ribogreen quantification (see Materials and Methods). For cDNA synthesis, an equal amount (ng) of total extracted RNA from the unpreserved and stabilization condition was used for a given urine sample. cDNA was prepared using the M-MLV Reverse Transcription kit and qPCR was performed using β-actin TaqMan assay (see Materials and Methods).

FIG. 10D (i) illustrates ΔCₜ which stands for [C_{t(T7)}-C_{t(T0)}] for β-actin (ACTB) RNA content in both unpreserved and stabilization solutions containing urine specimens after storage for 7 days at RT. An increase in ΔCₜ [ΔCₜ median value of ≥+3.5, FIG. 10C (i)] for β-actin (ACTB) RNA demonstrates loss of EV RNA content in unpreserved specimens stored for 7 days at room temperature. Chem F containing specimens showed median ΔCₜ value of +1.1 for β-actin RNA demonstrating efficient stabilization of EV RNA content after 7 days at room temperature. On the other hand, despite showing a median ΔCₜ value of +1.8 for β-actin RNA for 7 days stability time point (T7), Streck preservative containing urine specimens showed significant loss of EV RNA at Day 0 time point (median ΔCₜ value of +3.3) suggesting overall loss of EV RNA stability and content [FIG. 10D (ii)].

TABLE 20
Composition of the present invention prior to mixture with urine.

| Composition | Stock solution |
|---|---|
| Sodium acetate | 1750 mM |
| Boric acid | 5% |
| CDTA | 119 mM |
| Fructose | 45% |
| pH | 4.7-5.0 |

TABLE 21
Final composition of the present invention after mixture with urine.

| | Stabilizing |
|---|---|
| Composition | Solution (Chem F) |
| Sodium acetate | 233.3 mM |
| Boric acid | 0.67% |
| CDTA | 15.9 mM |
| Fructose | 6.0% |
| Ethanol | 6.3% |

TABLE 22 (i)
Composition of the present invention prior to mixture with urine.

| Composition | Stock solution |
|---|---|
| Sodium acetate | 771.2 mM |
| Boric acid | 2.2% |
| CDTA | 52.4 mM |
| Fructose | 19.8% |
| Ethanol | 22.4% |
| pH | 5.0 |

TABLE 22 (ii)
Final composition of the present invention after mixture with urine.

| | Stabilizing Solution |
|---|---|
| Composition | (Chem F) |
| Sodium acetate | 231.4 mM |
| Boric acid | 0.67% |
| CDTA | 15.7 mM |
| Fructose | 5.9% |
| Ethanol | 6.7% |

TABLE 22 (iii)
Composition of the present invention prior to mixture with urine.

| | Stock solution with | Stock solution with |
|---|---|---|
| Composition | Fructose (Chem F) | Glucose (Chem G) |
| Sodium acetate | 750 mM | 750 mM |
| Boric acid | 2.2% | 2.2% |
| CDTA | 50 mM | 50 mM |
| Sugar | 20% | 20% |
| Ethanol | 23% | 23% |
| pH | 5.0-5.2 | 5.0-5.2 |

TABLE 22 (iv)
Final composition of the present invention after mixture with urine.

| | Stabilizing Solution | Stabilizing Solution |
|---|---|---|
| Composition | (Chem F) | (Chem G) |
| Sodium acetate | 225 mM | 225 mM |
| Boric acid | 0.7% | 0.7% |
| CDTA | 15 mM | 15 mM |
| Sugar | 6% | 6% |
| Ethanol | 6.9% | 3.9% |

TABLE 23 (i)
Composition of the present invention prior to mixture with urine.

| Composition | Stock solution |
|---|---|
| Sodium acetate | 750 mM |
| Boric acid | 2.2% |
| CDTA | 50 mM |
| Fructose | 20% |
| Ethanol | 23% |
| pH | 5.0-5.2 |

TABLE 23 (ii)
Final composition of the present invention after mixture with urine.

| | Stabilizing Solution |
|---|---|
| Composition | (Chem F) |
| Sodium acetate | 225 mM |
| Boric acid | 0.7% |
| CDTA | 15 mM |
| Fructose | 6% |
| Ethanol | 6.9% |

### Example 11: Stabilizing Composition for the Preservation of Cell-Free RNA (cfRNA) in Urine at Room Temperature

Male and Female healthy donors provided random (mid-day), first void urine samples using the Colli-Pee® First Void Urine Collection Device (Novosanis). Specimens were transported to the laboratory on ice packs where samples were pooled together to form pooled urine specimens (MP, male-pooled; FP, female-pooled). An aliquot of pooled urine was stored in the 1) absence of a stabilizing composition (unpreserved), 2) mixed with Chemistry F stabilizing composition in the urine: chemistry ratio of 1:0.43 and stored. The composition of the stabilization solution is described in Table 24. Both types of specimens were stored at room temperature (23±3° C.) for at least 7 days. The final composition of the stabilization solution after mixing with urine is described in Table 25. On day zero and day 7, 2.5 mL aliquot of each unpreserved and Chem F containing urine specimen was centrifuged at 3,000 g for 10 minutes at room temperature, followed by 0.8 μM filtration. 2 mL of precleared supernatant was recovered from each specimen post-centrifugation and filtration and cell-free nucleic acids were extracted using the QiaAmp circulating nucleic acids extraction kit (Qiagen, see Materials and Methods). Extracted nucleic acids were subjected to DNAse digestion to remove DNA contamination for the efficient purification of total cell-free RNA. The concentration of extracted RNA samples was measured using Ribogreen quantification (see Materials and Methods). For cDNA synthesis, an equal amount (ng) of total extracted RNA from the unpreserved and stabilization condition was used for a given urine sample. cDNA was prepared using the M-MLV Reverse Transcription kit and qPCR was performed using β-actin TaqMan assay (see Materials and Methods).

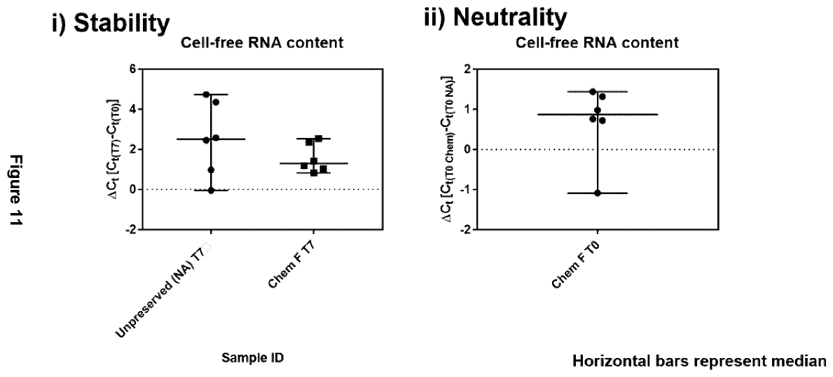

FIG. 11(i) illustrates ΔCₜ which stands for [C_{t(T7)}-C_{t(T0)}] for β-actin (ACTB) RNA content in both unpreserved and Chem F containing urine specimens after storage for 7 days at RT. There was an increase in ΔCₜ median value (+2.5) suggesting decrease in cell-free RNA content after the unpreserved specimens were stored for 7 days at room temperature; however, in contrast, there was less change in β-actin cell-free RNA levels in Chem F containing specimens after 7 days at room temperature as shown by ΔCₜ median value of 1.3 [FIG. 11(i)]. Moreover, there was no marked change in the cell-free RNA content in the urine samples upon addition of the chemistries relative to unpreserved(NA) samples at the time of collection (day 0) [FIG. 11 (ii)].

TABLE 24
Composition of the Stock Solution

| Composition | Stock Solution |
|---|---|
| Sodium acetate | 750 mM |
| Boric acid | 2.2% |
| CDTA | 50 mM |
| Fructose | 20% |
| Ethanol | 23% |
| pH | 4.7-5.0 |

TABLE 25
Final concentration of present composition after mixing with urine.

| | Stabilizing Solution |
|---|---|
| Composition | (Chem F) |
| Sodium acetate | 225 mM |
| Boric acid | 0.7% |
| CDTA | 15 mM |
| Fructose | 6% |
| Ethanol | 6.9% |

### Example 12: Stabilizing Composition for the Preservation of Urinary Cellular RNA in Urine at Room Temperature

This example is comprised of two separate studies. In the first study, mid-day first void urine samples from 8 male and 8 female donors were collected using the Colli-Pee® First-void Urine Collection Device (Novosanis) and pooled to form a total of 4 pooled urine specimens (2 male (MP) and 2 female (FP)). i) 40 mL of each specimen was stored in the absence of a stabilizing composition (unpreserved), and ii) 28 mL of each urine specimen was mixed with 12 mL of stock solution (Table 26). Both types of specimens were stored at room temperature (23±3° C.) for at least 7 days. The final composition after mixing with urine is described in Table 27 below.

In the second study, 4 healthy donors provided a 30 mL first morning, first void (FMFV) urine specimen and their urine samples were pooled together to generate two pooled urine specimens. i) 30 mL of each specimen was stored in the absence of a stabilizing composition (unpreserved, NA), and ii) 24 mL of each urine specimen was mixed with 4 mL of stock solution (Table 28) and 2 mL of 95% ethanol. Both types of specimens were stored at room temperature (23±3° C.) for at least 7 days. The final composition of the stabilization solution “Chem F” after mixing with urine is described in Table 29 below. For comparison, 25 mL of urine was mixed with 5 mL of Streck's urine fixative (reference composition), commercially known as “Cell-free DNA Urine Preserve” (Cat. No. 230216), and stored at room temperature for at least 7 days. The reference composition comprises the formaldehyde-releasing agent imidazolidinyl urea, as well as K₃EDTA and glycine.

On day zero and day 7, 15-16 mL aliquot of each unpreserved, Chem F and Streck's preservative containing urine specimen was centrifuged at 3,800 g for 20 minutes at room temperature. Total cellular pellet was recovered from each specimen post-centrifugation and urinary cellular RNA was extracted using either Trizol LS reagent (study I) as described in the Materials and Methods or Qiagen RNeasy plus Mini Kit (study II) according to manufacturer's protocol. Targeted mRNA analysis on extracted cellular RNA using β-actin (ACTB) TaqMan based RT-qPCR experiments was performed as described (see Materials and Methods).

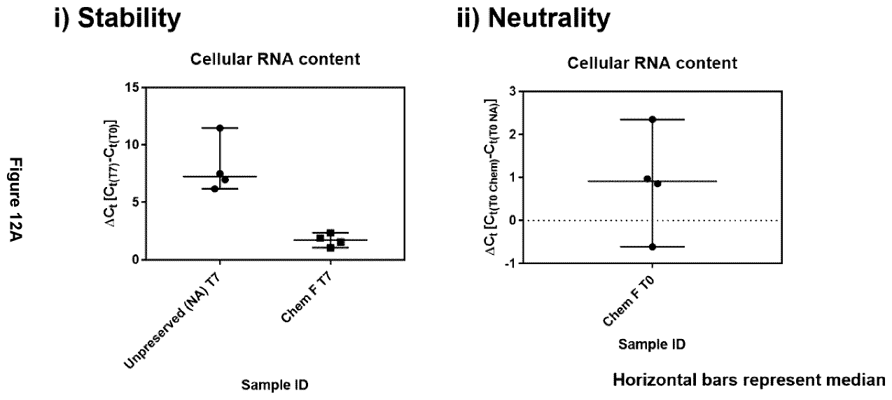

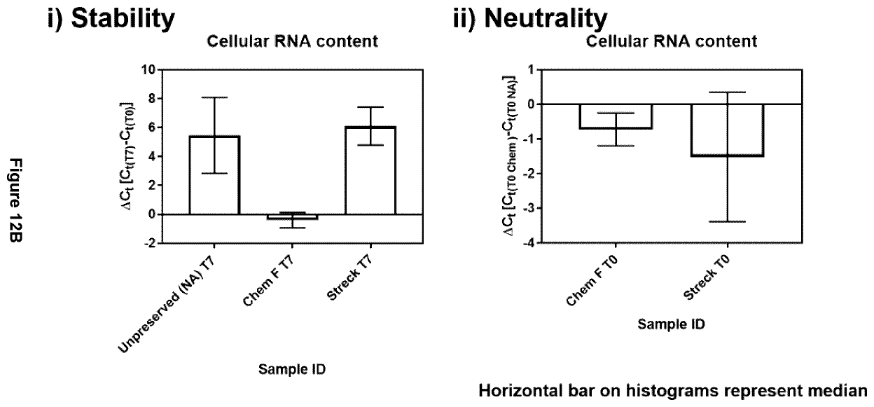

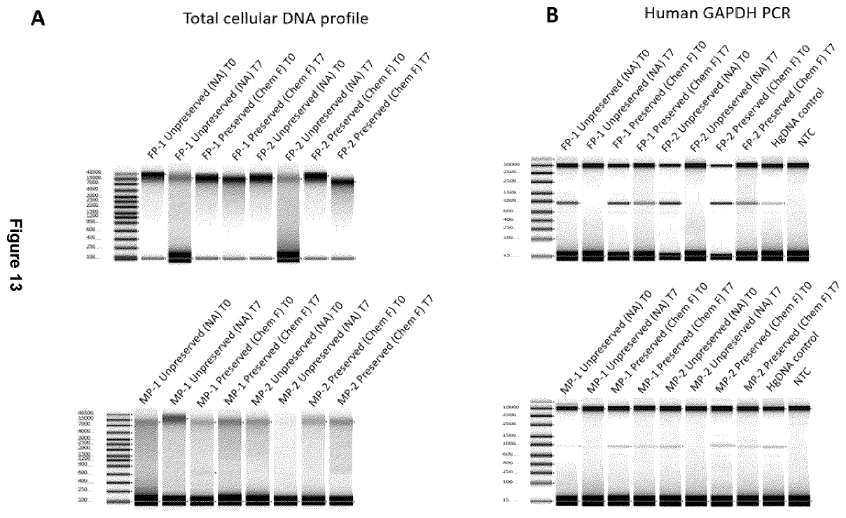

Overall data from the total of 4 samples (2 female-pooled and 2 male-pooled urine samples) from first experimental set-up is combined and presented in FIG. 12A. FIG. 12A(i) illustrates ΔCₜ which stands for [C_{t(T7)}-C_{t(T0)}] for β-actin (ACTB) RNA content in both unpreserved and Chem F containing urine specimens after storage for 7 days at RT. There was a dramatic increase in ΔCₜ for cellular β-actin (ACTB) RNA content demonstrating drastic loss of cellular RNA content in the unpreserved specimens stored for 7 days at room temperature. However, ΔCₜ for cellular β-actin RNA was significantly lower in Chem F containing specimens when compared to unpreserved specimens (FIG. 12A(i)) which indicates cellular RNA stability in the urine specimens containing stabilization solution after 7 days at room temperature. Moreover, there was no major change in the cellular RNA content in the urine samples upon addition of the chemistries relative to unpreserved(NA) samples at the time of collection (day 0) [FIG. 12A (ii)].

FIG. 12B(i) further shows a dramatic increase in ΔCₜ for cellular β-actin (ACTB) RNA content demonstrating a drastic loss of cellular RNA content in both the unpreserved, as well as Streck's urine preservative-containing specimens stored for 7 days at room temperature. However, ΔCₜ for cellular β-actin was significantly lower in Chem F containing specimens when compared to unpreserved and Streck's preservative containing specimens [FIG. 12B(i)]. Moreover, when compared to Chem F containing specimens, the Streck's preservative containing specimen showed more change in the cellular RNA content in urine samples upon addition of the chemistries relative to unpreserved (NA) samples at the time of collection (day 0) [FIG. 12B(ii)]. Overall, this data indicates cellular RNA stability in specimens containing the present stabilizing composition after 7 days at room temperature.

TABLE 26
Composition of the present invention prior to mixture with urine.

| Composition | Stock solution |
|---|---|
| Sodium acetate | 771.2 mM |
| Boric acid | 2.2% |
| CDTA | 52.4 mM |
| Fructose | 19.8% |
| Ethanol | 22.4% |
| pH | 5.0 |

TABLE 27
Final composition of the present invention after mixture with urine.

| | Stabilizing Solution |
|---|---|
| Composition | (Chem F) |
| Sodium acetate | 231.4 mM |
| Boric acid | 0.67% |
| CDTA | 15.7 mM |
| Fructose | 5.9% |
| Ethanol | 6.7% |

TABLE 28
Composition of the present invention prior to mixture with urine.

| Composition | Stock Solution |
|---|---|
| Sodium acetate | 1750 mM |
| Boric acid | 5% |
| CDTA | 119 mM |
| Fructose | 45% |
| pH | 4.7-5.0 |

TABLE 29
Final composition of the present invention after mixture with urine.

| | Stabilizing Solution |
|---|---|
| Composition | (Chem F) |
| Sodium acetate | 233.33 mM |
| Boric acid | 0.67% |
| CDTA | 15.87 mM |
| Fructose | 6% |
| Ethanol | 6.3% |

### Example 13: Stabilizing Composition for the Preservation of Urinary Cellular DNA in Urine at Room Temperature

In this study, mid-day first void urine samples from 6 male and 6 female healthy donors were collected using the Colli-Pee® First-void Urine Collection Device (Novosanis) and pooled to form a total of 4 pooled urine specimens [2 male-pooled (MP) and 2 female-pooled (FP)]. i) 30 mL of each specimen was stored in the absence of a stabilizing composition (unpreserved), and ii) 21 mL of each urine specimen was mixed with 9 mL of stock solution (Table 30). Both types of specimens were stored at room temperature (23±3° C.) for at least 7 days. The final composition after mixing with urine is described in Table 31 below.

On day zero and day 7, 15 mL aliquot of each unpreserved and Chem F containing specimen was centrifuged at 3000 g for 10 minutes at room temperature. Total cellular pellet was recovered from each specimen post-centrifugation and urinary cellular DNA was extracted using QiaAmp DNA Mini Kit (Qiagen) according to manufacturer's protocol. The profile of the extracted cellular DNA was assessed on Agilent 4200 Tapestation using Genomic DNA tape. Extracted DNA was used to amplify ˜1 Kb PCR product (GAPDH gene) for measuring DNA stability as described (see Materials and Methods).

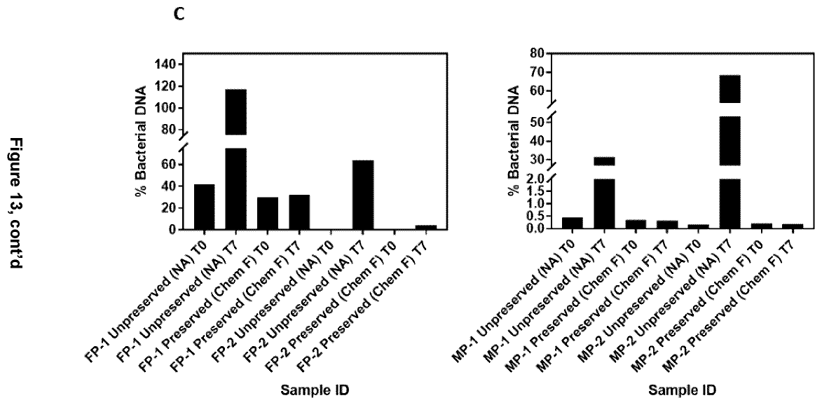

FIG. 13A illustrates the Tapestation profile of day 0 and day 7 extracted cellular DNA in both unpreserved (NA) and Chemistry F containing urine specimens. In FP samples, there was a consistent dramatic loss of high molecular weight genomic DNA in the unpreserved specimens stored for 7 days at room temperature. In MP unpreserved samples, one pooled sample showed an increase in high molecular weight genomic DNA due to bacterial growth, while the second pooled sample showed a significant decrease in high molecular weight genomic DNA after 7 days at room temperature. However, the profile of high molecular weight genomic DNA was preserved (FIG. 13A) in both Chem F containing FP and MP urine specimens after 7 days at room temperature; thus indicating cellular DNA stability. Next, targeted amplification of GAPDH gene for an amplicon size of ˜1 Kb was performed to determine the stability of high molecular weight DNA band in both the unpreserved and Chem F containing urine specimens at day 0 and day 7 time points.

FIG. 13B shows results of GAPDH PCR amplification. The presence of ˜1 Kb product strongly demonstrates human cellular DNA stability in both the Chem F containing FP and MP urine specimens after storage for 7 days at room temperature. GAPDH PCR amplification failed in the unpreserved specimens indicating lack of human cellular DNA stability. Bacterial 16S qPCR was performed on the DNA extracted from both the FP and MP specimens as described in the Materials and Methods. Bacterial 16s qPCR showed dramatic increase in the percentage of bacterial DNA content in both the FP and MP unpreserved urine samples kept for 7 days at RT; unlike chemistry F (Chem F) containing specimens which showed no significant change in the bacterial DNA content at day 7 relative to day 0 (FIG. 13C). Overall, the data suggests preservation of human cellular DNA and prevention of bacterial growth in the urine specimens containing stabilization solution after storage at room temperature for 7 days. On the other hand, unpreserved specimens showed complete loss of human cellular DNA and a dramatic increase in the bacterial DNA after storage at room temperature for 7 days.

TABLE 30
Composition of the present invention prior to mixture with urine.

| Composition | Stock Solution |
|---|---|
| Sodium acetate | 771.2 mM |
| Boric acid | 2.2% |
| CDTA | 52.4 mM |
| Fructose | 19.8% |
| Ethanol | 22.4% |
| pH | 5.0 |

TABLE 31
Final composition of the present invention after mixture with urine.

| | Stabilizing Solution |
|---|---|
| Composition | (Chem F) |
| Sodium acetate | 231.4 mM |
| Boric acid | 0.67% |
| CDTA | 15.7 mM |
| Fructose | 5.9% |
| Ethanol | 6.7% |

### Example 14: Stabilizing Composition for the Preservation of Cell-Free Nucleic Acids Profile in Saliva Samples Stored at Room Temperature

Like urine, saliva sampling is easy, safe, inexpensive and ideal for in-home collection. Saliva is composed of various molecules (e.g. enzymes, hormones, antibodies, mucins, growth factors, nucleic acids, exosomes, and antimicrobial constituents) that are filtered, processed and secreted from the vasculature that nourish the salivary glands. Many of these enter saliva from blood by passing through the spaces between cells by transcellular or para-cellular routes. Therefore, most compounds found in blood are also present in saliva. Hence, saliva shows high potential for monitoring health and disease (Y-H Lee and D T Wong (2009) Saliva: an emerging biofluid for early detection of diseases. Am J Dent 22(4): 241-248; K-A Hyun, H Gwak, J Lee, B Kwak, H-I Jung (2018) Salivary exosome and cell-free DNA for cancer detection. Micromachines 9: 340).

In this study, raw saliva samples were collected from 6 healthy individuals and were mixed together to form a pooled saliva sample. i) 7 mL of pooled saliva sample was mixed with 3 mL of 1× TE buffer (Thermo Fisher Scientific; Cat. No. AM9858) (unpreserved), and ii) 7 mL of pooled saliva was mixed with 3 mL of stock solution (see Table 32). TE buffer was added to the unpreserved specimen, to overcome the mucinous nature of saliva for the efficient separation of extracellular and cellular compartments. Both types of specimens were stored at room temperature (23±3° C.) for at least 7 days. The final composition after mixing with saliva is described in Table 33 below.

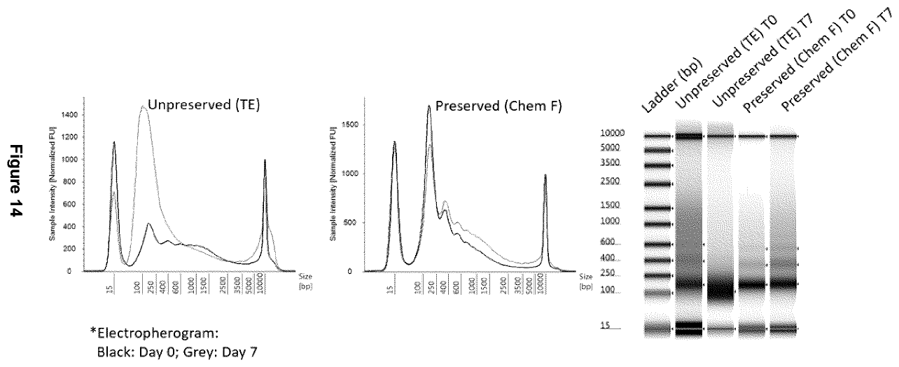

On day zero and day 7, 4.5 mL aliquot of each unpreserved and chemistry F (Chem F) containing specimen was centrifuged at 3,800 g for 20 minutes at room temperature. 4.0 mL of supernatant was recovered from each specimen post-centrifugation and cell-free nucleic acids were extracted using the QIAamp Circulating Nucleic Acids Kit (Qiagen, see Materials and Methods). The profile of the extracted cell-free nucleic acids was assessed on Agilent 4200 Tapestation using HS D5000 tape (Agilent; Cat. No. 5067-5592). FIG. 14 illustrates the Tapestation profile of day 0 and day 7 extracted cell-free DNA in both unpreserved (NA) and preserved Chem F containing saliva specimens. Tapestation data (FIG. 14) clearly demonstrates the preservation of cell-free DNA profile in saliva sample with stabilization solution after 7 days at room temperature, while there was a dramatic change in the cell-free DNA profile in the unpreserved sample after 7 days at room temperature when compared to day 0 profile.

TABLE 32
Composition of the present invention prior to mixture with saliva

| Composition | Stock Solution |
|---|---|
| Sodium acetate | 771.2 mM |
| Boric acid | 2.2% |
| CDTA | 52.4 mM |
| Fructose | 19.8% |
| Ethanol | 22.4% |
| pH | 5.0 |

TABLE 33
Final composition of the present invention
after mixture with saliva.

| | Stabilizing Solution |
|---|---|
| Composition | (Chem F) |
| Sodium acetate | 231.4 mM |
| Boric acid | 0.67% |
| CDTA | 15.7 mM |
| Fructose | 5.9% |
| Ethanol | 6.7% |

All publications, patents and patent applications mentioned in this Specification are indicative of the level of skill of those skilled in the art to which this invention pertains and are herein incorporated by reference to the same extent as if each individual publication, patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims. The scope of the claims should not be limited to the preferred embodiments set for the description, but should be given the broadest interpretation consistent with the description as a whole.

1. An aqueous stabilizing composition for preserving a bodily fluid at ambient temperature, the composition comprising:
a sugar selected from a monosaccharide, a disaccharide, or a combination thereof;
a buffering agent;
a C₁-C₆ alkanol;
boric acid, a salt of boric acid, or a combination thereof; and
a chelating agent;
wherein the composition has a pH of from 4.5 to 5.2.

2. The composition of claim 1, wherein the sugar is a monosaccharide.

3. The composition of claim 2, wherein the monosaccharide is selected from fructose, glucose, mannose, galactose, or a combination thereof.

4. The composition of claim 3, wherein the monosaccharide is selected from fructose, glucose, or a combination thereof.

5. The composition of claim 1, wherein the sugar is a disaccharide.

6. The composition of claim 5, wherein the disaccharide is selected from trehalose, lactose, or sucrose, or a combination thereof.

7. The composition of claim 6, wherein the disaccharide is sucrose.

8. The composition of any one of claims 1-7, wherein the buffering agent is acetate buffer, citrate buffer, or a combination thereof;
optionally, wherein the acetate buffer is selected from sodium acetate, potassium acetate, ammonium acetate, or a combination thereof;
optionally, wherein the citrate buffer is selected from sodium citrate, ammonium citrate, or a combination thereof.

9. The composition of claim 8, wherein the buffering agent is sodium acetate.

10. The composition of any one of claims 1-9, wherein the C₁-C₆ alkanol is selected from methanol or ethanol.

11. The composition of claim 10, wherein the C₁-C₆ alkanol is ethanol.

12. The composition of any one of claims 1-11, wherein the chelating agent is selected from ethylenediaminetriacetic acid (EDTA), 1,2-cyclohexanediamine tetraacetic acid (CDTA), diethylenetriamine pentaacetic acid (DTPA), tetraazacyclododecanetetraacetic acid (DOTA), tetraazacyclotetradecanetetraacetic acid (TETA), desferioximine, or chelator analogs thereof.

13. The composition of claim 12, wherein the chelating agent is CDTA.

14. The composition of any one of claims 1-13, wherein:
the sugar is present in an amount of from about 5% to about 45% (wt/vol), of from about 5% to about 40% (wt/vol), or from about 10% to about 30% (wt/vol), or from about 18% to about 22% (wt/vol), or about 20% (wt/vol),
the buffering agent is present in an amount of from about 150 mM to about 1.75 M, or from about 150 mM to about 1.5 M, or from about 500 mM to about 1.2 M, or from about 0.7 M to about 0.8 M, or about 0.75 M;
the C₁-C₆ alkanol is present in an amount of from about 5% to about 50% (vol/vol), or from about 10% to about 30% (vol/vol), or from about 20% to about 25% (vol/vol), or about 23% (vol/vol);
the boric acid, the salt of boric acid or the combination thereof is present in an amount of from about 0.5% to about 5% (wt/vol), or from about 1% to about 3% (wt/vol), or from about 2% to about 2.5% (wt/vol), or about 2.2% (wt/vol), and
the chelating agent is present in an amount of from about 10 mM to about 120 mM, or from about 10 mM to about 100 mM, or from about 30 mM to about 70 mM, or from about 40 mM to about 60 mM, or about 50 mM.

15. The composition of any one of claims 1-14, wherein the composition comprises, consists essentially of, or consists of:
fructose, glucose, sucrose, or a combination thereof in an amount of from about 5% to about 45% (wt/vol), or from about 5% to about 40% (wt/vol), or from about 10% to about 30% (wt/vol), or from about 18% to about 22% (wt/vol), or about 20% (wt/vol),
sodium acetate in an amount of from about 150 mM to about 1.75 M, or from about 150 mM to about 1.5 M, or from about 500 mM to about 1.2 M, or from about 0.7 M to about 0.8 M, or about 0.75 M;
methanol, ethanol, or a combination thereof in an amount of from about 5% to about 50% (vol/vol), or from about 10% to about 30% (vol/vol), or from about 20% to about 25% (vol/vol), or about 23% (vol/vol);
boric acid in an amount of from about 0.5% to about 5% (wt/vol), or from about 1% to about 3% (wt/vol), or from about 2% to about 2.5% (wt/vol), or about 2.2% (wt/vol), and
CDTA in an amount of from about 10 mM to about 120 mM, or from about 10 mM to about 100 mM, or from about 30 mM to about 70 mM, or from about 40 mM to about 60 mM, or about 50 mM.

16. The composition of any one of claims 1-15, wherein the composition comprises, consists essentially of, or consists of:
fructose, glucose, or a combination thereof in an amount of from about 5% to about 45% (wt/vol), or from about 5% to about 40% (wt/vol), or from about 10% to about 30% (wt/vol), or from about 18% to about 22% (wt/vol), or about 20% (wt/vol),
sodium acetate in an amount of from about 150 mM to about 1.75 M, or from about 150 mM to about 1.5 M, or from about 500 mM to about 1.2 M, or from about 0.7 M to about 0.8 M, or about 0.75 M;
ethanol in an amount of from about 5% to about 50% (vol/vol), or from about 10% to about 30% (vol/vol), or from about 20% to about 25% (vol/vol), or about 23% (vol/vol);
boric acid in an amount of from about 0.5% to about 5% (wt/vol), or from about 1% to about 3% (wt/vol), or from about 2% to about 2.5% (wt/vol), or about 2.2% (wt/vol), and
CDTA in an amount of from about 10 mM to about 120 mM, or from about 10 mM to about 100 mM, or from about 30 mM to about 70 mM, or from about 40 mM to about 60 mM, or about 50 mM.

17. The composition of any one of claims 1-16, wherein the composition stabilizes cells, extracellular vesicles, nucleic acids, and/or microorganisms contained in the bodily fluid.

18. The composition of claim 17, wherein the cells are selected from cancer cells or nucleated blood cells.

19. The composition of claim 17, wherein the nucleic acid is deoxyribonucleic acid (DNA).

20. The composition of claim 19, wherein the DNA comprises cell-free DNA (cfDNA), such as circulating tumor DNA (ctDNA).

21. The composition of claim 17, wherein the nucleic acid is ribonucleic acid (RNA).

22. The composition of claim 21, wherein the RNA comprises cell-free RNA (cfRNA).

23. The composition of claim 21, wherein the RNA comprises extracellular vesicle RNA (EV RNA).

24. The composition of claim 17, wherein the microorganisms are selected from bacteria or viruses.

25. A method for preserving a bodily fluid, the method comprising:
a) obtaining a sample of the bodily fluid;
b) contacting the bodily fluid with an aqueous stabilizing composition as defined in any one of claims 1-24 to form a mixture;
c) mixing the mixture of (b) to form a homogeneous mixture; and
d) storing the homogeneous mixture at ambient temperature.

26. The method of claim 25, wherein preserving the bodily fluid comprises stabilizing cells, extracellular vesicles, nucleic acids, and/or microorganisms contained in the bodily fluid.

27. The method of claim 26, wherein the cells are selected from cancer cells or nucleated blood cells.

28. The method of claim 26, wherein the nucleic acid is deoxyribonucleic acid (DNA).

29. The method of claim 28, wherein the DNA comprises cell-free DNA (cfDNA), such as circulating tumor DNA (ctDNA).

30. The method of claim 26, wherein the nucleic acid is ribonucleic acid (RNA).

31. The method of claim 30, wherein the RNA comprises cell-free RNA (cfRNA).

32. The method of claim 30, wherein the RNA comprises extracellular vesicle RNA (EV RNA).

33. The method of claim 26, wherein the microorganisms are selected from bacteria or viruses.

34. The method of any one of claims 26-33, wherein the cells, nucleic acids, extracellular vesicles, and/or microorganisms contained in the bodily fluid are stabilized for at least 7 days at ambient temperature, or for at least 14 days at ambient temperature.

35. The composition of any one of claims 1-24, or the method of any one of claims 25-34, wherein the bodily fluid is urine or saliva.

36. An aqueous composition comprising:
a sugar selected from a monosaccharide, a disaccharide, or a combination thereof;
a buffering agent;
a C₁-C₆ alkanol;
boric acid, a salt of boric acid, or a combination thereof;
a chelating agent; and
a bodily fluid.

37. The composition of claim 36, wherein the sugar is a monosaccharide.

38. The composition of claim 37, wherein the monosaccharide is selected from fructose, glucose, mannose, galactose, or a combination thereof.

39. The composition of claim 38, wherein the monosaccharide is selected from fructose, glucose, or a combination thereof.

40. The composition of claim 36, wherein the sugar is a disaccharide.

41. The composition of claim 40, wherein the disaccharide is selected from trehalose, lactose, or sucrose, or a combination thereof.

42. The composition of claim 41, wherein the disaccharide is sucrose.

43. The composition of any one of claims 36-42, wherein the buffering agent is acetate buffer, citrate buffer, or a combination thereof;
optionally, wherein the acetate buffer is selected from sodium acetate, potassium acetate, ammonium acetate, or a combination thereof;
optionally, wherein the citrate buffer is selected from sodium citrate, ammonium citrate, or a combination thereof.

44. The composition of claim 43, wherein the buffering agent is sodium acetate.

45. The composition of any one of claims 36-44, wherein the C₁-C₆ alkanol is selected from methanol or ethanol.

46. The composition of claim 45, wherein the C₁-C₆ alkanol is ethanol.

47. The composition of any one of claims 36-46, wherein the chelating agent is selected from ethylenediaminetriacetic acid (EDTA), 1,2-cyclohexanediamine tetraacetic acid (CDTA), diethylenetriamine pentaacetic acid (DTPA), tetraazacyclododecanetetraacetic acid (DOTA), tetraazacyclotetradecanetetraacetic acid (TETA), desferioximine, or chelator analogs thereof.

48. The composition of claim 47, wherein the chelating agent is CDTA.

49. The composition of any one of claims 36-48, wherein:
the sugar is present in an amount of from about 1.5% to about 15% (wt/vol), or from about 2% to about 10% (wt/vol), or from about 5% to about 7% (wt/vol), or about 6% (wt/vol),
the buffering agent is present in an amount of from about 50 mM to about 500 mM, or from about 200 mM to about 400 mM, or from about 220 mM to about 240 mM, or about 230 mM, or about 225 mM;
the C₁-C₆ alkanol is present in an amount of from about 2% to about 40% (vol/vol), or from about 3% to about 20% (vol/vol), or from about 5% to about 10% (vol/vol), or about 6.5% (vol/vol), or about 6.9% (vol/vol);
the boric acid, the salt of boric acid or the combination thereof is present in an amount of from about 0.1% to about 2% (wt/vol), or from about 0.2% to about 1.5% (wt/vol), or from about 0.5% to about 1.0% (wt/vol), or about 0.7% (wt/vol), or about 0.6% (wt/vol); and
the chelating agent is present in an amount of from about 2.5 mM to about 50 mM, or from about 5 mM to about 25 mM, or from about 10 mM to about 20 mM, or about 16 mM, or about 15 mM.

50. The composition of any one of claims 36-49, wherein the bodily fluid is urine or saliva.


SEQUENCE LISTING (10 sequences)

SEQ ID NO: 1 - DNA, 17 bp, Artificial Sequence
  artificially synthesized sequence
attaccgcggctgctgg

SEQ ID NO: 2 - DNA, 17 bp, Artificial Sequence
  artificially synthesized sequence
cctacgggaggcagcag

SEQ ID NO: 3 - DNA, 20 bp, Artificial Sequence
  artificially synthesized sequence
acacaactgtgttcactagc

SEQ ID NO: 4 - DNA, 20 bp, Artificial Sequence
  artificially synthesized sequence
caacttcatccacgttcacc

SEQ ID NO: 5 - DNA, 22 bp, Artificial Sequence
  artificially synthesized sequence
aagatgtttttttgtgattggt

SEQ ID NO: 6 - DNA, 22 bp, Artificial Sequence
  artificially synthesized sequence
ttcctatttttactcacccaaa

SEQ ID NO: 7 - DNA, 19 bp, Artificial Sequence
  artificially synthesized sequence
agccataccaaacgacgag

SEQ ID NO: 8 - DNA, 19 bp, Artificial Sequence
  artificially synthesized sequence
agcaataaaccagccagcc

SEQ ID NO: 9 - DNA, 21 bp, Artificial Sequence
  artificially synthesized sequence
gtcaacggatttggtcgtatt

SEQ ID NO: 10 - DNA, 21 bp, Artificial Sequence
  artificially synthesized sequence
ctctcttcctcttgtgctctt